(19) United States
(12) Patent Application Publication
(10) Pub. No.: US 2021/0069697 A1
(43) Pub. Date: Mar. 11, 2021

(54) SYSTEMS AND METHODS FOR INTEGRATING SENSORS WITH PUMPS IN A MICROFLUIDIC DEVICE

(71) Applicant: Charles Stark Draper Laboratory, Inc., Cambridge, MA (US)

(72) Inventors: Hesham Azizgolshani, Belmont, MA (US); Jonathan Robert Coppeta, Windham, NH (US); Joseph L. Charest, Jamaica Plain, MA (US); Alex M. Zorn, East Boston, MA (US); Keith B. Baldwin, Amesbury, MA (US)

(21) Appl. No.: 17/013,456

(22) Filed: Sep. 4, 2020

Related U.S. Application Data
(60) Provisional application No. 62/896,349, filed on Sep. 5, 2019.

(51) Int. Cl. B01L 3/00

(52) U.S. Cl. CPC B01L 3/5085; B01L 2300/021; B01L 2300/0645; B01L 2300/0829

(57) ABSTRACT

This disclosure provides systems and methods for integrating an array of electronic sensors capable of performing trans-epithelial electrical resistance (TEER) measurements into a microfluidic device that includes a well plate. In some implementations, the sensors can include electrodes that are submerged into fluidically connected wells of the microfluidic device, which can contain an electrically conductive fluid such as the cell culture media or a buffered salt solution. An array of such electrodes can be integrated into a lid of the system that includes the microfluidic device. These electrodes can be routed using a printed circuit board through a number of multiplex switches that can allow addressing of a desired unit of the device through a microprocessor in communication with a computer.

### CROSS REFERENCE TO RELATED APPLICATIONS

The present application claims priority under 35 U.S.C. § 119(e) to U.S. Provisional Patent Application No. 62/896349, titled “SYSTEMS AND METHODS FOR INTEGRATING SENSORS WITH PUMPS IN A MICROFLUIDIC DEVICE,” filed Sep. 5, 2019, which is incorporated herein in its entirety by reference.

### BACKGROUND

Microfluidic devices can include features such as channels, chambers, and wells. It can be challenging to accurately measure cultured cells in controlled conditions.

### SUMMARY

The present disclosure describes systems and methods for integrating sensors with pumps in a microfluidic device. The microfluidic device can include features such as wells, chambers, or channels, at least some of which can be used for culturing a respective group of cells. In some implementations, the microfluidic device can include up to 96 wells, up to 384 wells, or any other number of wells, which may be arrayed in a rectangular grid pattern or any other type of pattern. Wells may be interconnected via channels of one or more microfluidic devices. Other active and passive components can also be included. For example, the microfluidic device can include or can interact with a series of microfluidic pumps that can be controlled to introduce fluid into the wells.

In some implementations, it may be desirable to measure or monitor certain conditions within the microfluidic device. For example, electronic measurements such as trans-epithelial electrical resistance (TEER) measurements can be useful for evaluating cell cultures within the microfluidic device. However, due to the potentially large number of wells and channels, as well other components (e.g., microfluidic pumps, fluid reservoirs, optical equipment, etc.), it can be challenging to integrate sensors into the microfluidic device for performing TEER measurements. This disclosure provides techniques for integrating an array of electronic sensors capable of performing TEER measurements into a microfluidic device that includes a well plate. In some implementations, the sensors can include electrodes that are submerged into the fluidically connected wells of the microfluidic device, which can contain an electrically conductive fluid such as the cell culture media or a buffered salt solution. An array of such electrodes can be integrated into a lid of the system that includes the microfluidic device. These electrodes can be routed using a printed circuit board through a number of multiplex switches that can allow addressing of a desired unit of the device through a microprocessor in communication with a computer.

At least one aspect of the present disclosure generally relates to a system. The system can include a well plate having one or more wells. The system can include a plurality of probes having a source electrode and a respective sense electrode. The plurality of probes can be disposed within the one or more wells of the well plate. The system can include a controller device. The system can receive an identification of a well of the well plate for a measurement. The system can select a probe of the plurality of probes that corresponds to the identification of the well for measurement. The system can establish a connection from a sensor to the source electrode and the respective sense electrode of the probe. The system can determine a measurement of a fluid sample in the well from the probe using the sensor. The system can store the measurement in memory.

In some implementations, the system can include one or more pumps. Each of the one or more pumps can be disposed within a respective well of the one or more wells. The one or more pumps can be electrically coupled to and controlled by the controller device. In some implementations, the source electrode and the respective sense electrode are each coupled to a respective pump. In some implementations, the system can include an airtight enclosure enclosing the controller device. In some implementations, the system can include a source of dry gas passing through the airtight enclosure to remove moisture from the controller device.

In some implementations, the one or more wells of the well plate can number up to 96 wells arranged in a rectangular pattern. In some implementations, the controller device can include one or more connectors configured to couple the controller device to the one or more pumps through mechanical force. In some implementations, the one or more connectors can include at least one of a spring device, a conductive flexible material, or a deformable material that is pushed into place against the one or more pumps. In some implementations, the source electrode and the sense electrode of each of the one or more probes can include at least one of silver, silver chloride, platinum, stainless steel, a polyimide polymer, or a polyether imide polymer.

In some implementations, the system can include a printed circuit board in electrical communication with the controller device and the one or more probes. In some implementations, the printed circuit board can include one or more multiplex switches. In some implementations, the system can establish the connection from a sensor to the source electrode and the respective sense electrode by addressing the selected probe using the one or more multiplex switches. In some implementations, the one or more connectors can each include an opening configured to receive a portion of a pump of the one or more pumps, and a plurality of cantilevers arranged around the opening of the connector and configured to electrically couple the portion of the pump to the connector.

At least one other aspect of the present disclosure is generally directed to a method. The method can be performed, for example, by a controller device in electrical communication with a plurality of probes. The method can include receiving an identification of a well of a well plate for a measurement. The method can include selecting a probe of the plurality of probes that corresponds to the identification of the well for measurement. The method can include establishing, via one or more switches, a connection from a sensor to a source electrode and a respective sense electrode of the probe. The method can include determining a measurement of the fluid sample in the well using the sensor. The method can include storing the measurement in a memory.

In some implementations, selecting the probe of the plurality of probes that corresponds to the identification of the well for measurement can include selecting a second source electrode and a second respective sense electrode. In some implementations, establishing the connection from the sensor to the probe can include electrically coupling the sensor to the source electrode and the respective sense electrode of the probe, such that the sensor can receive one or more signals from the probe. In some implementations, the sensor is integrated with the controller device, and determining the measurement of the fluid sample in the well can include providing a current source from the probe of the plurality of probes that corresponds to the identification of the well to a second probe of the plurality of probes. In some implementations, the method can include sensing, from the sense electrode of the probe, a voltage value between the sense electrode of the probe and the second probe of the plurality of probes. In some implementations, the method can include determining a resistance of a tissue sample in the well using the voltage value.

At least one other aspect of the present disclosure generally relates to a system. The system can include a plurality of current source electrodes of a first respective plurality of pump sippers disposed within a first respective plurality of wells in a well plate. The system can include a plurality of voltage sense electrodes of a second respective plurality of pump sippers disposed within a second respective plurality of wells. The second respective plurality of wells can be coupled to the first plurality of wells via a channel comprising a solution including cells. The system can include a controller device. The system can route an electric current through the cells in the channel using a first current source electrode and a second current source electrode of the plurality of current source electrodes. The system can measure a voltage level across the cells caused in part by the electric current. The system can determine a parameter of the solution using the voltage level. The system can actuate a pump for one of the first plurality of pump sippers or the second plurality of pump sippers based at least on the parameter.

In some implementations, the system can determine an impedance of the solution including the cells as the parameter. In some implementations, the system can generate one or more signals to one or more switches to cause the electric current to flow through the first current source, the cells, and the second current source. In some implementations, the plurality of current source electrodes and the plurality of voltage sense electrodes can include at least one of round wires, flat wires, conductive tubes, or multi-lumen tubes.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the invention can be combined with other aspects. Aspects can be implemented in any convenient form.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. The foregoing and other objects, aspects, features, and advantages of the disclosure will become more apparent and better understood by referring to the following description taken in conjunction with the accompanying drawings, in which:

FIG. 1 a sectional view of an example system for integrating sensors with a microfluidic device, in accordance with an illustrative embodiment;

FIG. 2 illustrates a top view of an example well plate, in accordance with an illustrative embodiment;

FIG. 3 illustrates a top view of an example microfluidic channels of an example well plate, in accordance with an illustrative embodiment;

FIG. 4 illustrates a perspective view of a fluidic circuit that can be used in the system of FIG. 1, in accordance with an illustrative embodiment;

FIG. 5 illustrates a schematic view of an example circuit that can serve as an electronic sensor in the system of FIG. 1, in accordance with an illustrative embodiment;

FIG. 6A illustrates a top view of a connector that can be used for routing signals used by each electrode of the circuit shown in FIG. 5, in accordance with an illustrative embodiment;

FIG. 6B ill illustrates a perspective view of the connector of FIG. 6A coupled with an electrode of the circuit of FIG. 5, in accordance with an illustrative embodiment;

FIG. 7 illustrates a top view of an electrical routing board including a plurality of connectors similar to the connectors of FIGS. 6A and 6B, in accordance with an illustrative embodiment;

FIG. 8 illustrates views of the electrical routing board of FIG. 7 coupled with electrodes and a digital multiplex board, in accordance with an illustrative embodiment;

FIG. 9 illustrates a perspective view of the electrical routing board and the digital multiplex board of FIG. 8 within an enclosure, in accordance with an illustrative embodiment;

FIG. 10 illustrates a top view of the digital multiplex board shown in FIG. 8, in accordance with an illustrative embodiment;

FIG. 11 illustrates a top view of the electrical routing board shown in FIG. 7, in accordance with an illustrative embodiment;

FIG. 12 illustrates a block diagram of an example system used to perform the routing functionality as described herein, in accordance with an illustrative embodiment;

FIG. 13 illustrates a flow diagram of an example method of establishing connections to probes and measuring properties of microfluidic devices, in accordance with an illustrative embodiment;

FIG. 14 shows the general architecture of an illustrative computer system that may be employed to implement any of the computer systems discussed herein, in accordance with an illustrative embodiment;

FIGS. 15A and 15B depict example diagrams of the various systems described herein, in accordance with one or more implementations;

FIG .16 illustrates a perspective view of a fluidic circuit that can be used in the systems described herein, in conjunction with one or more connectors for pump sippers, in accordance with an illustrative embodiment;

FIG. 17 depicts an expanded view of the system depicted in FIG. 9 integrated with a housing and a well plate, in accordance with an illustrative embodiment;

FIGS. 18A and 18B depict example implementations of portions of the systems described herein, in accordance with one or more implementations;

FIG. 19A depicts a bottom view of an example implementation of a well plate for sensor calibration, in accordance with one or more implementations; and

FIG. 19B depicts a top view of an example implementation of a well plate for sensor calibration, in accordance with one or more implementations.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The present disclosure describes systems and methods for integrating sensors with pumps in a microfluidic device. The microfluidic device can include features such as wells, chambers, or channels, at least some of which can be used for culturing a respective group of cells. In some implementations, the microfluidic device can include 96 wells, 384 wells, or any other number of wells, which may be arrayed in a rectangular grid pattern. Wells may be interconnected via channels in the microfluidic device. Other active and passive components can also be included. For example, the microfluidic device can include or can interact with a series of microfluidic pumps that can be controlled to introduce fluid into the wells.

In some implementations, it may be desirable to measure or monitor certain conditions within the microfluidic device. For example, electronic measurements such as trans-epithelial electrical resistance (TEER) measurements can be useful for evaluating cell cultures within the microfluidic device. However, due to the potentially large number of wells and channels, as well other components (e.g., microfluidic pumps, fluid reservoirs, optical equipment, etc.), it can be challenging to integrate sensors into the microfluidic device for performing TEER measurements. This disclosure provides techniques for integrating an array of electronic sensors capable of performing TEER measurements into a microfluidic device that includes a well plate. In some implementations, the sensors can include electrodes that are submerged into the fluidically connected wells of the microfluidic device, which can contain an electrically conductive fluid such as the cell culture media or a buffered salt solution. An array of such electrodes can be integrated into a lid of a system that includes the microfluidic device. These electrodes can be routed using a printed circuit board through a number of multiplex switches that can allow addressing of a desired unit of the device through a microprocessor in communication with a computer.

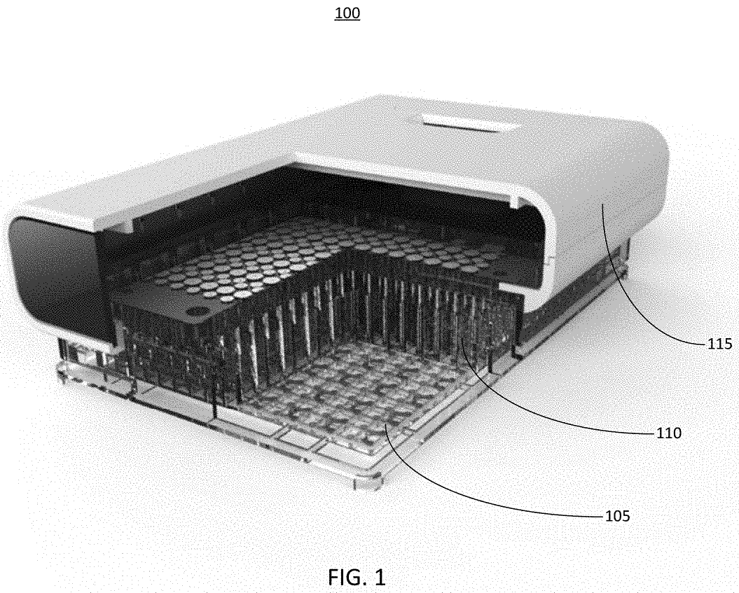

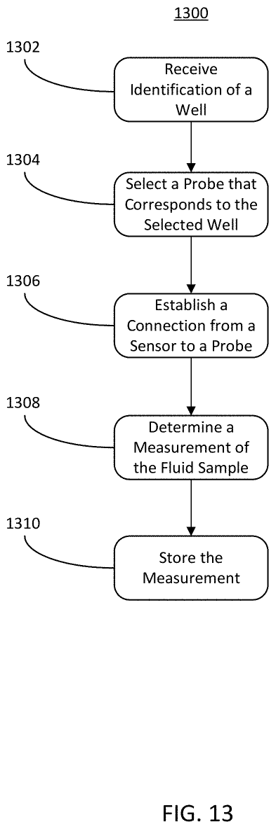

FIG. 1 illustrates a sectional view of an example system 100 for integrating sensors with a microfluidic device. The system 100 can include a housing 115. The housing 115 can be an enclosure that surrounds or partially surrounds other components of the system 100. The system 100 can include a microfluidic well plate 105. The well plate 105 can include a plurality of wells, which may be interconnected by a network of channels within the well plate 105. The system 100 can also include a series of microfluidic pumps 110. Each pump 110 can be coupled with a respective port defined by the well plate 105. Thus, the pumps 110 can control the introduction of fluid samples into the wells of the well plate 105 via the ports with which the pumps 110 are coupled. Each of the pumps can be electrically coupled to a controller, for example the controller 1205 described herein below in conjunction with FIG. 12.

In some implementations, the system 100 may also include additional or different components than those depicted in FIG. 1. For example, the system 100 can include electronic sensors, such as current sensors, voltage sensors, or any other type of electronic sensor or transducer. The sensors can be configured to perform TEER measurements of cell cultures within the well plate 105. For example, a component of TEER can include impedance, which can be measured using one or more current sensors or one or more voltage sensors. In some implementations, the sensors can be integrated together (e.g., a voltage sensor integrated with a current sensor, etc.). In some implementations, the sensors can be integrated with the pumps 110. For example, each pump 110 can include a respective sensor or portion of a sensor for performing TEER measurements. In some implementations, the system 100 can also include associated electronic components to control the pumps 110, to apply power to the sensors, and to analyze outputs of the sensors. At least some of these electronic components can be enclosed within the housing 115. For example, the electronic components can be positioned above the pumps 110 inside the housing 115.

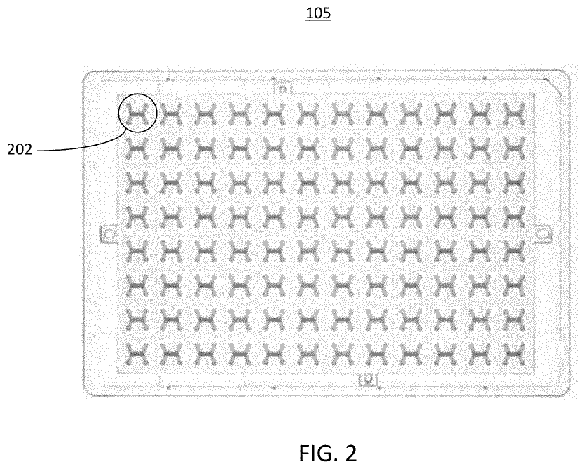

FIG. 2 illustrates a top view of an example well plate 105 that can be used in the system 100 depicted in FIG. 1. As shown in the figure, the wells of the well plate 105 can be coupled to a series of microfluidic devices 202 (sometimes referred to as cell culture unit(s) 202). In some implementations, the microfluidic devices 202 can comprise one or more channels with inlets and outlets that are coupled to the wells of the well plate 200. The microfluidic device 202 can include two or more channels that share an overlapping region. The implementation illustrated in FIG. 2 includes microfluidic devices 202 having two overlapping channels, where each overlapping channel has an inlet port and an outlet port. In some implementations, the inlet port and the outlet ports of the microfluidic devices can be bidirectional (e.g., serve as either an inlet port or an outlet port, etc.). The ports of the microfluidic device can be formed as part of the wells of the well plate 105, or may be coupled to the wells of the well plate through adhesion, mechanical coupling, or other coupling means. In implementations where each microfluidic device 202 has two channels that each have two ports, each microfluidic device 202 can be coupled to four wells on the well plate 105. Each of the four wells can be coupled to a respective one of the ports of the microfluidic device 202.

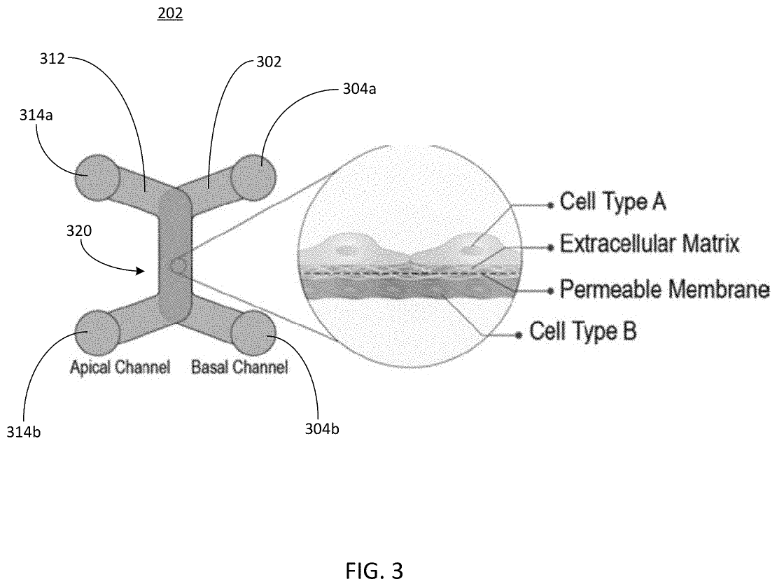

The microfluidic devices 202 can be arranged in a pattern to accommodate the wells of the well plate 105, or any other openings of substrates in implementations where a well plate is not present. The arrangement of the channels of each microfluidic device 202 can change to accommodate the shape of the wells or openings of the well plate 105, or any other substrate to which the microfluidic device is coupled. In FIG. 2, the well plate 105 is depicted as including 96 microfluidic devices 202 arranged in a 12 by 8 grid, however it should be understood that other arrangements, which may include more or fewer microfluidic devices 202, are also possible. In some implementations, the well plate 105 can have more or fewer wells or openings, which may be arranged differently than what is depicted in FIG. 2. Further, in some implementations, each channel of the microfluidic device 202 may have more than or fewer than two openings. For example, in some implementations, a microfluidic device 202 may have channels each having one opening, where fluid flow is facilitated through each channel via the overlapping region of the channels. Each microfluidic device 202 can serve as an area for one or more cell cultures to be introduced, for example by cell seeding techniques. After the cell cultures have been introduced to and grown in the microfluidic device 202, the systems and methods of this disclosure can take measurements of the cultures in the microfluidic devices via the ports of the well plate 105. In some implementations, other substances, such as therapeutic substances, can be introduced into the microfluidic devices 202, and their interactions with the cell cultures can be observed or measured. The structure of the microfluidic device 202 is described in further detail below in conjunction with FIG. 3.

Referring now to FIG. 3, depicted is a top view of an example microfluidic device. The microfluidic device 202 can have at least one channel having one or more openings, which can be a part of or coupled to the well plate 105 described herein above in conjunction with FIG. 2. The channels of the microfluidic device may be formed from any suitable material to facilitate the flow of fluid, for example a plastic substrate, a metal substrate, a polymer substrate, a ceramic material, a composite material substrate, or any other type of material suitable for forming microfluidic channels. In some implementations, the microfluidic device 202 can be formed as a part of a well plate, such as the well plate 105 described herein above in conjunction with FIG. 2. The microfluidic device 202 or the components thereof may be formed by various processes, including wet etching, reactive ion etching, conventional machining, photolithography, soft lithography, injection molding, laser ablation, in situ construction, plasma etching, or any combination thereof.

The microfluidic device 202 can include a basal channel 302. The basal channel can include two ports 304a and 304b (generally referred to as ports 304), each of which can serve as either an inlet port, an outlet port, or both (e.g., each port may serve as either an inlet port or an outlet port, etc.). The ports 304 of the basal channel 302 can be openings in the channel 302 that are configured to receive a fluid, such as a fluid containing cells for a cell seeding or culturing process. The ports 304 can receive other devices, such as portions of pumps, pipettes, sippers, probes, current probes, voltage probes, or other measurement devices. Although the basal channel 302 is depicted as having two ports, it should be understood that the ports 304 can be any number of ports (e.g., one port 304, two ports 304, three ports 304, etc.). The ports 304 can be coupled to one or more wells of the well plate 105, or can be formed as a part of one or more wells of the well plate 105. In some implementations, the ports 304 are openings to the basal channel 302, and are not coupled to a well plate or formed as a part of a well plate. Thus, the microfluidic device 202 can exist in a number of different configurations, such as part of a well plate, or coupled to a different type of device for cell culturing or measurement.

The microfluidic device 202 can include an apical channel 312. The apical channel 312 can include two ports 314a and 314b (generally referred to as ports 314). The ports 314 of the apical channel 312 can be openings in the channel 302 that are configured to receive a fluid, such as a fluid containing cells for a cell seeding or culturing process. Although the apical channel 312 is depicted as having two ports, it should be understood that the ports 314 can be any number of ports (e.g., one port 304, two ports 314, three ports 314, etc.). The ports 314 can be coupled to one or more wells of the well plate 105, or can be formed as a part of one or more wells of the well plate 105. The ports 314 can receive other devices, such as portions of pumps, pipettes, sippers, probes, current probes, voltage probes, or other measurement devices. In some implementations, the ports 314 are openings to the apical channel 312, and are not coupled to a well plate or formed as a part of a well plate. .

The microfluidic device 202 can include an overlapping portion 320 (sometimes referred to as an overlapping region 320) in which the basal channel 302 and the apical channel 312 overlap one another. By way of non-limiting example, the basal channel 302 can be disposed beneath the apical channel 312, and the overlapping region 320 can form a top wall of the basal channel 302, and a bottom wall of the apical channel 312. Thus, the overlapping portion 320 can form a portion of each of the apical channel 312 and the basal channel 302. In some implementations, the apical channel 312 and the basal channel 302 can be disposed differently with respect to one another, but share an overlapping region that forms a portion of either channel. In some implementations, the overlapping portion 320 can be formed as part of the microfluidic device, or may be disposed among one or more layers of the microfluidic device. In some implementations, the overlapping portion 320 can be fixed in place by mechanical force, an adhesive, or formed as part of at least one of the basal channel 302 or the apical channel 312.

The overlapping region 320 can be configured to trap and grow cell cultures, for example cell cultures or cells within a fluid sample that passes through at least one of the apical channel 312 or the basal channel 302. The overlapping portion 320 can be porous, or otherwise semipermeable, thus facilitating flow of one or components of a fluid between the basal channel 302 and the apical channel 312, or vice versa. The overlapping portion 320 can be made of materials other than the materials that define the basal channel 302 or the apical channel 312. For example, the overlapping portion can be or include any of a membrane (e.g., a semipermeable membrane, etc.), a filter, a mesh, or any other substance that allows some or all of a fluid to pass through the overlapping portion 320. Thus, the overlapping portion 320 can facilitate the flow of a fluid sample between the basal channel 302 and the apical channel 312, while trapping cells in the fluid sample on the respective portion of the membrane within one of the apical channel 312 or the basal channel 302. Thus, the overlapping portion 320 can be seeded with cells that can be grown into a culture. By using sensors inserted into one or more of the ports 304 or the ports 314 of the microfluidic device 202, parameters of the cell culture or another fluid sample in the microfluidic device 202 can be measured.

The overlapping portion 320 can include a cell scaffold such as a permeable membrane, as shown in the enlarged view on the right of FIG. 3. The scaffold can, at least in part, separate the basal channel 302 from the apical channel 312 in the overlapping portion 320. In some implementations, a cell culture (e.g., a fluid sample that includes one or more cells, etc.) can be introduced on the apical channel 312 side of the overlapping portion 320. In some implementations, a cell culture can be introduced on the basal channel 302 side of the overlapping portion 320. In some implementations, cell cultures can be introduced on both the apical channel 312 side and the basal channel 302 side of the overlapping portion 320. The cell cultures on each side of the scaffold can be the same or different from one another. In some implementations, fluid samples can be introduced into the basal channel 302 via the ports 304 and fluid samples can be introduced into the apical channel 312 via the ports 314. The fluid samples may include, for example, therapeutic substances such as drugs, cells, or any other type of particle or component. Thus, interactions between the cell cultures and the substances included in the fluid samples can be observed in the overlapping portion 320.

Although FIG. 3 is depicted as having two channels, it should be understood that the microfluidic device 202 can include any number of channels and overlapping regions 320. In implementations where the microfluidic device 202 has more than two channels, the overlapping regions 320 of can overlap with one or more of other channels in the microfluidic device 202, and can facilitate fluid flow between any of the channels while trapping cells contained in the fluid flow on one or more surfaces of the overlapping region 320.

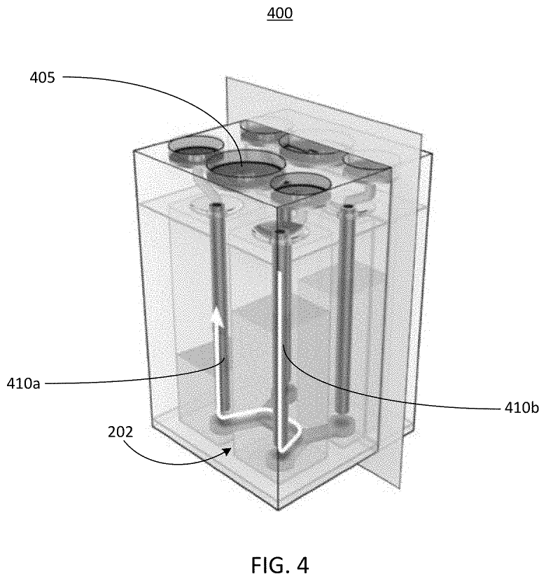

Referring now to FIG. 4, illustrated is a perspective view of a fluidic circuit 400 that can be used in the system of FIG. 1. The fluidic circuit 400 includes microfluidic reservoirs 405 coupled with pumps 410a and 410b (generally referred to as pumps 410). The pumps 410 can be the same as, or include all of the same functionality as, the pumps 110 described herein above in conjunction with FIG. 1. The microfluidic reservoirs can contain fluid samples that are introduced to the channels of the microfluidic device 202 by the pumps 410. The pumps 410 can have at least two ends, with one end coupled to at least one microfluidic reservoir and another end coupled to a portion of the microfluidic device 202, such as a port 304 or a port 314 described herein above in conjunction with FIG. 3. Thus, the pumps 410 can be coupled with a port of a well plate such as the well plate 105 shown in FIG. 1. For example, as depicted the pumps 410 may interface with a microfluidic device 202 similar to that shown in FIG. 2. In some implementations, each pump 410 may include a metal tube as depicted in FIG. 4, which can be referred to as a sipper. The sipper of each pump can deliver or remove fluid from a respective well of the well plate 105. In some implementations, the pumps 410 can create a pressure in the well plate that causes one or more fluid samples to pass through at least one channel of the microfluidic device 202. The sipper of each pump 410 can deliver fluid from the reservoirs 405 to the well plate below.

In some implementations, the pumps 410 can be microscale pumps. Microscale pumps can transport fluids in microliter volumes. For example, volumes of the reservoirs 405, the channels of the well plate, and the sippers of the pumps 410 can be in the microliter range (e.g., contain fluid samples on the order of microliters, etc.). In some implementations, the pumps 410 can be of a scale that is larger than the microscale range. For example, the volumes of the reservoirs 405, the channels of the well plate, and the sippers of the pumps 410 can contain fluid samples greater than the microliter range. In some implementations, the pumps 410 can be of a scale that is smaller than the microscale range. For example, the volumes of the reservoirs 405, the channels of the well plate, and the sippers of the pumps 410 can contain fluid samples smaller than the microliter range. The sippers of the pumps 410 can have lengths in the range of about 1 millimeter to about 10 millimeters. In some implementations, the sippers of the pumps 410 can have lengths that are longer than 10 millimeters, or shorter than 1 millimeter, or any range in between and including those values. In some implementations, the volume of the fluidic circuit 400 can be in the range of about 5 microliters to about 25 microliters. In some implementations, the volume of the fluidic circuit 400 (e.g., the path traced by the arrow from the pump 410a to the pump 410b, etc.) can be about 15 microliters. In some implementations, the volume of the fluidic circuit 400 can be greater than about 15 microliters. In some implementations, the volume of the fluidic circuit 400 can be less than about 15 microliters.

In some implementations, the fluidic circuit 400 can be actuated by electronics that control the pumps 410. For example, the pumps 410 can be controlled to achieve desired concentrations of compounds or other substances in the wells and channels of the well plate below, while also minimizing or reducing wasted compounds. The pumps 410 can be electrically coupled to and controller by a controller device, such as the controller 1205 described herein below in conjunction with FIG. 12. In some implementations, the sippers of each pump 410 can be formed from a conductive material. As a result, the sippers can be included as components of an electrical circuit. In some implementations, such an electrical circuit can form part of a sensor for performing TEER measurements. For example, each of the sippers of the pumps can be utilized as a probe, and can be routed to at least one current source or at least one voltage sensor using routing techniques as described herein. Thus, any of the pumps 410 can serve as an electrode for sensing various electrical properties of the fluids in the well plate 105 or in the microfluidic device 202. Depending on the type of sensor to which the conductive portions of the are coupled (e.g., by routing, etc.), the sippers can serve as at least one of a current source electrode, a voltage sense electrode, a ground electrode, a voltage source electrode, or any other type of electrode or sensing device. The conductive portions of the sippers can be routed to electrically couple to various sensors, including external sensors.

In some implementations, each pump 410 can be set or controlled to a condition to prevent significant current leak through the conductive fluid in the pump 410, before a TEER measurement is taken. For example, such a pump setting could ensure that a pump contains fluid but valves are closed to prevent current leak, to ensure that a pump 410 is pumped to a dry condition to remove the conductive fluid, or a combination of these two settings. The pump control and electrical data collection can be controlled by a common element, such as a processor, to coordinate their operation. A current leak can be an undesirable path taken by current. If fluid is present in the pump, or other conductive material forms a different circuit that present an unfavorable path (e.g., a path for current that does not pass through a target area for measurements, or a path that is longer than desired and passes through undesired areas, etc.). The actuation of valves and the various conditions of the pumps (e.g., pumping dry, pumping fluid, removing fluid, etc.) can be controlled by a controller device, such as the controller 1205 described herein below in conjunction with FIG. 12.

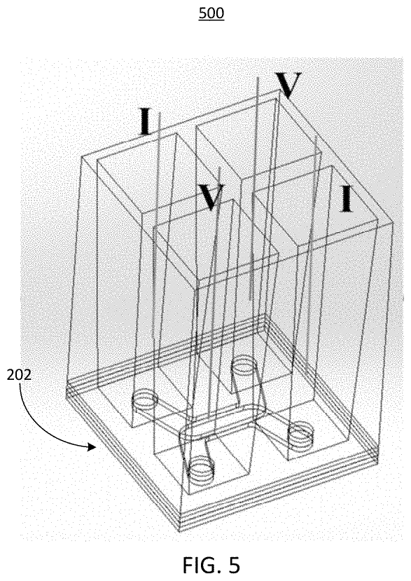

Referring now to FIG. 5, illustrated is a schematic view of an example circuit 500 that can serve as an electronic sensor in the system 100 of FIG. 1. For example, the circuit 500 can serve as a sensor (e.g., one or more probes gathering properties of a cells or fluid in a microfluidic device, etc.) for performing a TEER measurement on a particular microfluidic device 202. The circuit can comprise one or more electrodes inserted into the wells of the well plate 105 that are coupled to or form a part of the microfluidic device 202 under measurement (sometimes referred to as the target microfluidic device 202). In some implementations, the electrodes can be the sippers of the pumps 410 described herein above. To cause the electrodes to be part of the circuit 500, each of the electrodes can be routed (e.g., by the controller 1205 described herein below in conjunction with FIG. 12, etc.) using switching techniques to electrically coupled to a voltage sensor, a current sensor, a voltage source, or a current source, among others.

Generally, a TEER measurement can refer to an assay of the barrier function of the cultured cells within the microfluidic device 202 (e.g., the cells that are attached to and cultured on the overlapping region as described herein above, etc.). In this example, the TEER measurement can be achieved using a four point probe measurement which measures the electrical resistance of the tissue through providing two source electrodes and two sense electrodes. In the four point probe measurement, two of the electrodes can be current source electrodes, and two of the electrodes can be voltage sense electrodes. By changing the positioning (e.g., into which wells each electrode type is placed, etc.) of the current source electrodes and the voltage sense electrodes, different electrical paths are created through the microfluidic device 202. As shown in FIG. 5, a respective electrode can be positioned to a respective well coupled to or forming a part of one of the four ports of the microfluidic device 202. In some implementations, the electrodes can be submerged into the fluidically connected wells of the microfluidic device 202, which can contain an electrically conductive fluid such as the cell culture media or a buffered salt solution. The conductive fluid can aid in the creation of a path for current to flow through the cells, thus improving the accuracy of the TEER measurements.

While only a single circuit 500 including four probes is shown in FIG. 5, it should be understood that additional circuits similar to the circuit 500 can also be included in the system 100 of FIG. 1. In some implementations, each microfluidic device 202 of the system 100 can include a respective instance of the circuit 500. Thus, in implementations in which the system 100 includes 96 microfluidic devices 202, the system may also include 96 circuits 500. As each circuit 500 can include up to and including four electrodes, the system 100 may therefore include an array of up to and including 384 electrodes. However, it should be understood that more than or fewer than four electrodes may be used to measure the properties of fluids or cells within one or more of the microfluidic devices 202. In some implementations, the electrodes can be integrated into a lid or housing of the system 100, such as the housing 115 shown in FIG. 1.

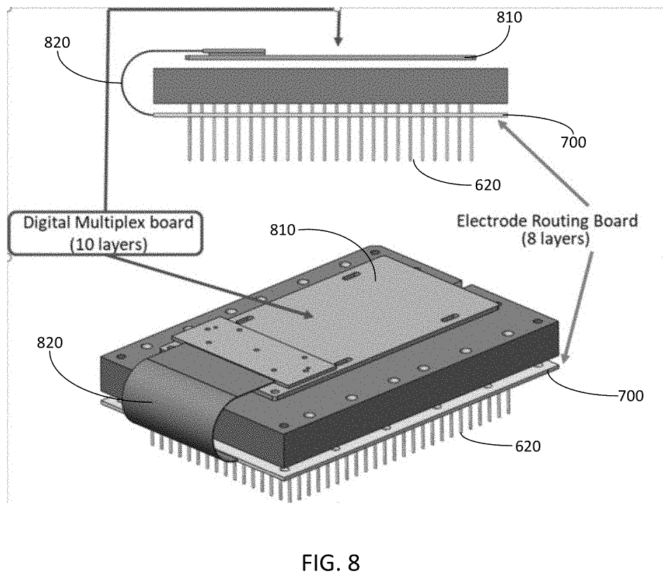

The electrodes can be electrically coupled to a switching board that can establish a connection between the probes and a sensor for analysis. In some implementations, the electrodes can be formed from one or more conductive sippers of the pumps 110 of the system 100. The sippers of the pumps that can control the fluid levels in each of the wells of the well plate 105 can be electrically conductive, and can be coupled to one or more sensors directly or via a switching board. Further details of the switching board are described herein below in conjunction with at least FIGS. 7-11. In implementations where the pump 410 sippers serve as the electrodes for the device, it should be understood that other electrodes that are different from the pump sippers can also be inserted into the wells, and connections can be established using the additional electrodes using the same processes as described herein. In some implementations, the four electrodes that can be used to acquire a four-point TEER measurement can be referred to as a probe. In some implementations, a probe may have fewer than four electrodes. For example, in some implementations, a probe may be referred to as any group of electrodes having at least one voltage sense electrode and at least one current source electrode.

In some implementations, the electrodes can be made or constructed in various forms including round or flat wires, tubes, or multi-lumen tubes. The electrodes can be made from various materials, such as to silver, silver chloride, platinum, stainless steel. In some implementations, the electrodes can be formed from metalized or other polymers treated to be conductive, such as polyimides or polyether imides. In some implementations, these electrodes can be routed using a printed circuit board through a number of multiplex switches that can allow addressing of a desired unit of the device (e.g., a particular instance of the circuit 500, associated with a respective instance of a microfluidic device 202) through a microprocessor in communication with a computer. Upon selection of the unit, the circuit 500 can be configured such that an externally connected measurement device containing a current source and a voltage sense, such as potentiostat, is routed through to the circuit 500 of interest. The resulting measurement can be recorded onto an external or internal memory device. In some implementations, the memory device can be part of the computer or other control unit that controls the circuit 500 or the configuration of the multiplex switches. In some other implementations, the circuit 500 can be integrated into the multiplexing board and the four point measurement can done using commercially available chips. In another embodiment, the circuit 500 and other components of the systems described in this disclosure can be used to perform electrical impedance spectroscopy.

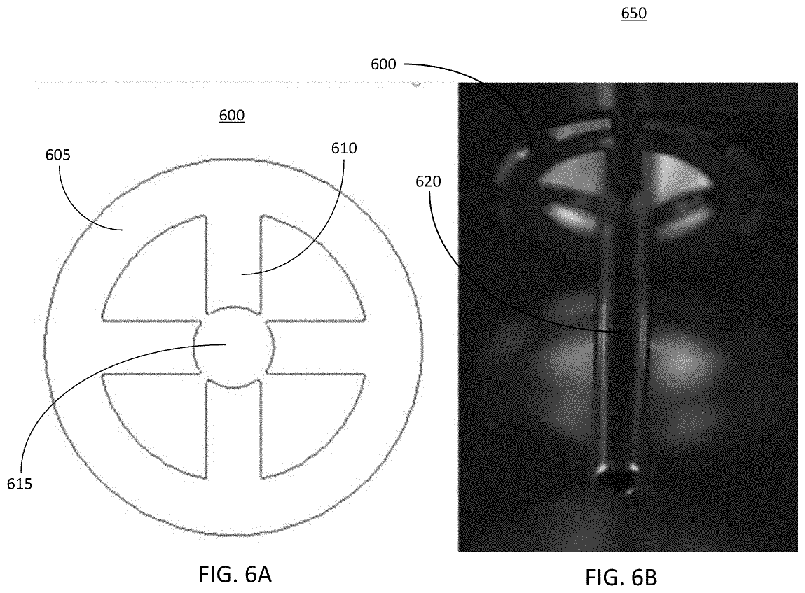

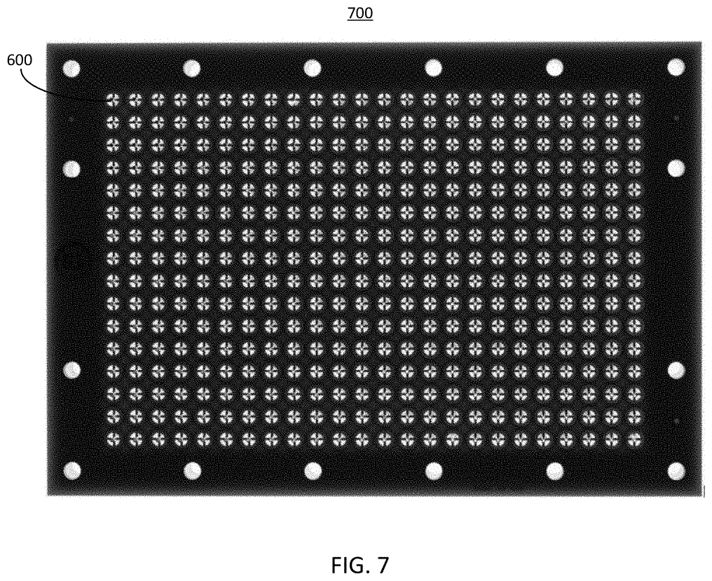

Referring now to FIG. 6A, illustrated is a top view of a connector 600 that can be used for routing signals used by each electrode of the circuit 500 shown in FIG. 5. In implementations where a pump sipper or other electrode is inserted into or received by a well of a well plate 105, the electrode must be electrically coupled to multiplex switches or to one or more sensors to be valuable for measurement. Thus, FIG. 6A illustrates a top view of the connectors 600 that interface with and couple to the electrodes in one or more of the wells of the well plate. For example, in some implementations, the sippers of the pumps 410 shown in FIG. 4 can serve as electrodes in the circuit 500 of FIG. 5. Each of the connectors can contact and electrically couple the respective electrodes to another circuit, for example a switching circuit or a sensor circuit.

In implementations where the pump 410 sippers serve as the electrodes that are used to take measurements of fluid samples or cells cultures of the microfluidic device 202, the connectors 600 can be configured to electrically couple with a respective one of the sippers to route the signal from the sipper elsewhere (e.g., via an electrical routing board coupled with the connector 600). Because the sippers of the pumps 410 are electrically conductive, they can serve as electrodes for use in measuring voltage, current, or providing a source of voltage or current. The connectors 600 can be configured to hold or otherwise mechanically couple to the electrodes. The connector 600 can include a ring 605 and a plurality of cantilevers 610 arranged as spokes in a circular fashion around an opening 615. In some implementations, the ring 605 can be permanently connected to the electrode routing board, for example as a component that is soldered to or otherwise affixed to the routing board. The opening 615 can be configured to receive the sipper of a corresponding pump. To receive the corresponding pump, the sipper of the pump can past through the opening such the sipper contacts one or more electrically conductive portions of the connector. In some implementations, the diameter of the opening 615 can be equal to or smaller than the outer diameter of the sipper. Thus, the cantilevers 610 can exert a mechanical contact force on the sipper to provide an electrical connection between the connector 600 and the sipper.

Referring now to FIG. 6B, illustrated is a perspective view of the connector of FIG. 6A coupled with an electrode of the circuit 500 of FIG. 5. In this example, the electrode 620 can be a sipper of a pump, which can be similar to the pumps 410 shown in FIG. 4. Thus, the electrodes 620 can sometimes be referred to as the sipper(s) 620 or the pump sipper(s) 620. The electrode 620 is inserted through the central opening 615 in the connector 600. The cantilevers 610 are contacted by the electrode 620 and deflect downward as a result. For example, the diameter of the opening 615 can be smaller than an outer diameter of the electrode 620 so that the electrode 620 is pressed against the cantilevers 610. Each of the cantilevers can be made of a conductive material, and can be electrically coupled to a switching board that can be used to route signals to and from the electrically conductive portion of the sipper. In this example, the sipper is constructed of a conductive material, and thus can be utilized as an electrode for various measurements of cell cultures or fluid samples of a microfluidic device 202, as described herein. Thus, in addition to guiding the pump sipper into a respective well of a well plate for requested measurements, the cantilevers of the connector 600 can electrically couple the pump sipper to other circuitry for accurate measurements.

In some implementations, the contact force of the connector 600 on the electrode 620 can be tuned by changing the diameter of the opening 615 and/or the diameter of the electrode 620 to adjust the interference between them, as well as by changing the width and thickness of the cantilevers 610. In some implementations, the connector 600 can have threads that are configured to engage with threads present on the electrode 620, thus forming electrical and mechanical contacts between sipper and the connector 600. To maintain modularity, the connector 600 can allow for removal of other electronic components (e.g., a digital multiplex board for controlling, receiving, and/or analyzing signals from the electrode 620) from the electrodes 620 themselves. Other implementations of electrical connectors similar to the connector 600 can include formed metal sheets that allow for connecting an electrical routing board to vertically oriented electrodes 620 at the side of the electrodes 620. In some implementations, a conductive feature (such as the connector 600 or another type of connector) can be welded to each electrode 620, for example via laser welding or other methods.

It should be understood that different geometries for the connector 600 are also possible. For example, the connector 600 can be or can include a circular or other shape opening that press-fits to the electrode 620, a spring device which presses against the electrode 620, a flexible material (e.g., a polymer) made of or containing conductive material that impinges an opening through which the electrode 620 protrudes, or a deformable material which is pushed into place against the electrode 620. The connector 600 can be formed, for example from a conductive metal, a conductive polymer, or a conductive alloy, in order to form an electrical connection with the electrode 620.

FIG. 7 illustrates a top view of an electrical routing board 700 including a plurality of connectors similar to the connectors 600 of FIGS. 6A and 6B. The electrical routing board 700 includes connectors 600 arranged in a rectangular array of 24 by 16 instances of the connectors 600. However, it should be understood that any number or arrangement of the connectors 600 are possible. In some implementations, the connectors 600 can be integrated with the routing board 700. The electrical routing board can be utilized in conjunction with a controller device, such as the controller 1205 described herein below in conjunction with FIG. 12, to establish electrical connections between one or more of the electrodes 620 and one or more sensors.

In some implementations, the layout of the connectors 600 on the electrical routing board 700 can match or correspond to the layout of the electrodes that form instances of the circuit 500 for performing TEER measurements, as shown in FIG. 5. Thus, in implementations in which the electrodes include sippers of pumps, the arrangement, layout, and spacing of the connectors 600 on the electrical routing board 700 can be the same as the arrangement, layout, and spacing of the sippers of the pumps. Further, the arrangement, layout, and spacing of the connectors 600, the pumps, and the electrodes can conform to the arrangement, layout, and spacing of the wells of the well plate 105 that serve as openings to the channels of the microfluidic devices 202. As a result, the sippers of the pumps can be coupled with respective connectors 600 substantially simultaneously by aligning the connectors 600 of the electrical routing board 700 with their respective sippers and pressing the electrical routing board 700 over the sippers to form the electrical connections between the sippers and their respective connectors 600. After passing through the connectors, the sippers of the pumps can be inserted into respective wells of the well plate 105, thus completing the fluidic circuit 400 shown in FIG. 4, and the electric measurement circuit 500 shown in FIG. 5.

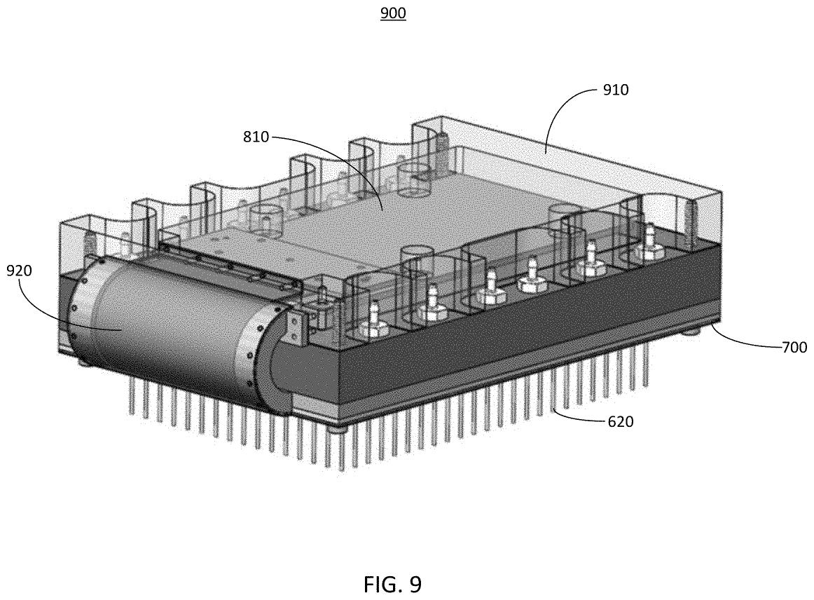

Referring now to FIG. 8, illustrated are views of the electrical routing board 700 of FIG. 7 coupled with electrodes 620 and a digital multiplex board 810. In FIG. 8, the electrical routing board 700 routing the electrical signals from the electrodes 620 can be a rigid-flex board that is connected to the tubing(s) of each pump (also referred to as sippers), for example via the connectors 600 shown in FIGS. 6A and 6B. The electrodes 620 can be formed from stainless steel or metalized or coated tubing to impart electrochemical properties to facilitate electrical connections. In some other implementations, the electrical routing board 700 can be coupled with the electrodes 620 by other means, such as through soldering, conductive epoxy, etc. The electrical routing board 700 can be coupled with the electrodes 620 either permanently (e.g., via soldering) or reversibly (e.g., using the connector 600 of FIGS. 6A and 6B).

The electrical routing board 700 can route signals or establish connections from the electrodes 620 to the top side of the pumps to move active electrical components away from the fluid filled plate. In some implementations, the electrical routing board 700 and the digital multiplex board 810 can be connected using a high density interconnect, such as the flexible interconnect 820 shown in FIG. 8. The flexible interconnect 820 can include many wires that electrically couple the digital multiplex board 810 to the electrical routing board 700. In some other implementations, the electrical routing board 700 and the digital multiplex board can be fabricated as one piece without the need for a connector. The digital multiplex board 810 can include one or more multiplex switches that can be actuated by digital signals, for example digital signals generated by a controller device such as the controller 1205 described herein below in conjunction with FIG. 12. The digital multiplex board can be configured to route the signals, which can be analog signals or digital signals, from the electrodes coupled to the electrical routing board 700 to one or more sensors. As described herein below, the one or more sensors can be external to the controller 1205 or internal to the controller 1205.

To electrically couple the connectors 600 on the electrical routing board 700, each of the connectors 600 can be electrically coupled (e.g., via printed circuit board routing on the electrical routing board 700, etc.) the flexible interconnect 820. In some implementations, the flexible interconnect 820 can include a wire or other conductive material that is for and electrically coupled to each the connectors 600 on the electrical routing board 700. The flexible interconnect can have at least two ends, one end terminating at and electrically coupled to the electrical routing board 700, and another end terminating at and electrically coupled to a connector that can be electrically coupled to the digital multiplex board 810. Thus, each of the electrodes 620 can be electrically coupled to the multiplex switching board 810, which can be configured to establish connections between any one of the electrodes 620 and one or more sensors. In some implementations, the one or more sensors can be present on the multiplex switching board 810. In other implementations, the one or more sensors can be external to the multiplex switching board 810, but may be connected to the multiplex switching board 810 via one or more connectors or communication networks.

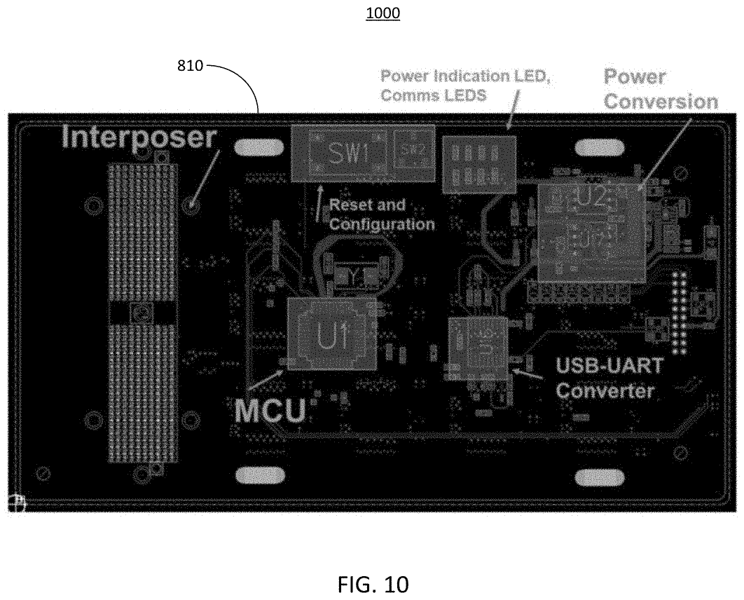

Referring now to FIG. 9, illustrated is a perspective view 900 of the electrical routing board 700 and the digital multiplex board 810 of FIG. 8 within an enclosure 910. The enclosure 910 can be an airtight enclosure. The enclosure 910 can prevent environmental damage to the electrical components of the electrical routing board 700 and the digital multiplex board 810. In some implementations, the enclosure 910 can include or can receive a supply of nitrogen or other dry gases, which may be passed through a portion of the enclosure 910 to remove moisture from the immediate environment of the electrical components of the electrical routing board 700 and the digital multiplex board 810. The enclosure 910 can include, or be a part of, the housing 115 described herein above in conjunction with FIG. 1. In some implementations, the enclosure 910 can also include a portion 920 that encloses the connector coupling the electrical routing board 700 and the digital multiplex board 810. In some implementations, the electrical routing board 700 and the digital multiplex board 810 can be surrounded or enclosed by a potting compound such as an epoxy, elastomer, or other potting material, in order to isolate the electrical routing board 700 and the digital multiplex board 810 from the external environment. The potting compound can be used in addition to or in place of the enclosure 910. In some implementations, the enclosure 910 can also enclose at least a portion of the pumps included in the device.

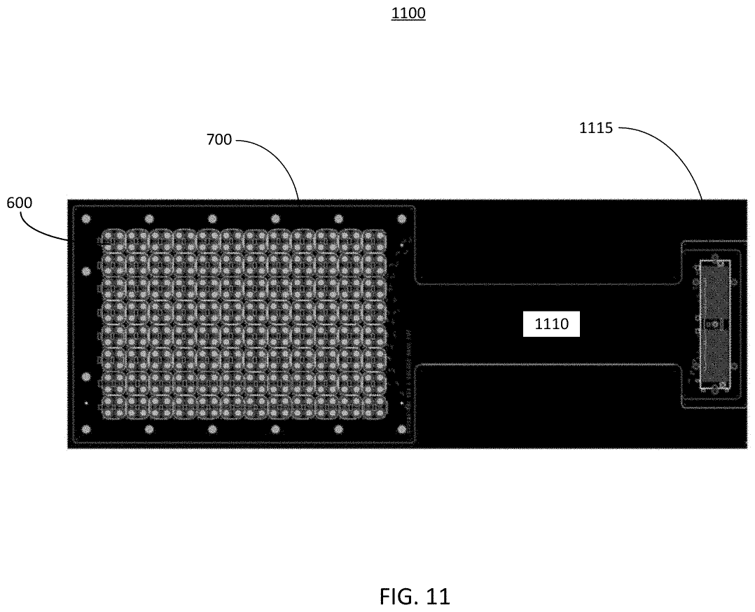

Referring now to FIG. 10, illustrated is a top view 1000 of the digital multiplex board 810 shown in FIG. 8. The digital multiplex board 810 can include indicators, such as a power indication light emitting diode (LED) and a communications LED. The power indication LED can indicate when the digital multiplex board 810 is receiving power. The power to the multiplex board can drive the various functionalities described herein, including the functionality of the controller 1205. The communications LED can indicate that one or more of the devices on the digital multiplex board 810 are communicating with another device. For example, the digital multiplex board 810 can include a USB to UART converter module. When the USB to UART module is communicating with another device, for example an external computing system, the USB to UART converter can cause the communication LED to illuminate on the digital multiplex board. In some implementations, the communication LED is on when power is received, and dims or temporarily turns off as information is communicated via one or more communications interfaces, such as the USB to UART converter.

The digital multiplex board 810 can include a power conversion module. The power conversion module can convert one or more power signals in to various voltage levels required by the components of the digital multiplex board 810, such as the MCU, the USB to UART converter, the interposer, or any other components as described herein. In some implementations, the power conversion module can be a step-up converter. In some implementations the power conversion module can be a step-down converter. In some implementations, the power conversion module can be a buck-boost converter that is capable of generating one or more DC voltage levels to power the circuitry of the digital multiplex board 810. The digital multiplex board 810 can include a reset and configuration module.

The reset and configuration module of the digital multiplex board can control the reset functionality of the MCU or other components of the digital multiplex board 810. For example, the reset and configuration module can include a button that, when actuated, causes the MCU or other components of the digital multiplexing board to reset. The digital multiplex board 810 can include an interposer. As described herein above, the interposer can electrically couple the electrodes inserted into the wells of a well plate 105 to the digital multiplexer board 810. The digital multiplexing board 810 can include a microcontroller unit (MCU). In some implementations, the microcontroller can be or be a part of the controller 1205 described herein below in conjunction with FIG. 12. The digital multiplexer board 810 can include various multiplexing switches that are configured to route a connection between a sensor and one or more of the electrodes 620 coupled to the electrical routing board 700. For example, the multiplex switches may be addressable, for example via in a computer address space. In some implementations, the digital multiplex switches can receive one or more commands via a communications interface of a computing device, such as the controller 1205 or the computing device 1400. In some implementations, a communications interface and transmit one or more actuation signals that can route a signal from a desired electrode to a desired sensor using the multiplex switches.

The multiplex switches can be arranged such that each of the electrodes 620 of the electrical routing board is addressable by a computing device. For example, the multiplex switches can be arranged such that appropriate actuation of each switch (e.g., selecting to turn the switch on or off) can cause an electrically conductive path between the desired electrode and a sensor. Each of the multiplex switches can have at least two states: an ON state and an OFF state. In the on state, the multiplex switch can conduct electricity (e.g., to create an electrical pathway). In the off state, the multiplex switch can be in a high-impedance state (e.g., does not create an electrical pathway). The multiplex switches can be arranged and connected on the multiplex board such that a proper combination of ON and an OFF state can cause a pathway to be created from each electrode to a sensor on or external to (e.g., attached via connector, etc.) the digital multiplex board. In some implementations, the multiplex switches can be arranged and connected on the multiplex board such that a proper combination of ON and an OFF state can cause a pathway to be created from each group of four electrodes (e.g., for a single TEER measurement of a microfluidic device 202, etc.) to a sensor on or external to (e.g., attached via connector, etc.) the digital multiplex board. The multiplex switches can change state in response to signals received from a controller device, such as the controller 1205 described herein below in conjunction with FIG. 12.

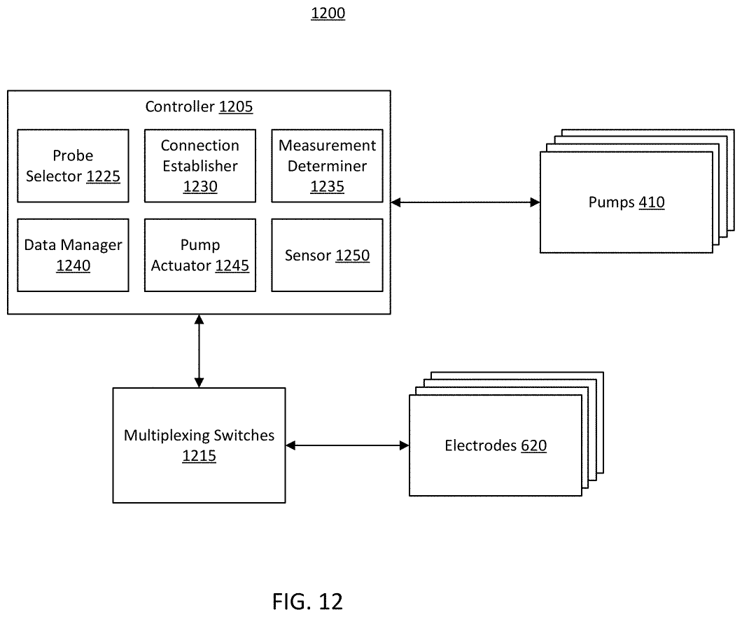

FIG. 11 illustrates a top view 1100 of the electrical routing board 700 shown in FIG. 7. The electrical routing board 700 can include a plurality of connectors 600, as depicted in FIG. 7. The electrical routing board 700 can include a high density connector 1115. The high density connector 1115 can be configured to interface with the digital multiplex board 810 via the interposer shown in FIG. 10. The electrical routing board 700 can include a ribbon cable 1110 coupling the electrical routing board 700 with the high density connector 1115. The ribbon cable 1110 can be a flexible cable containing at least one wire or electrically conductive element (e.g., filament, material, etc.) electrically coupling each of the electrodes 620 to a respective connector element on the high-density connector 1115.

Referring now to FIG. 12, illustrated is a block diagram of an example system 1200 used to perform the routing functionality as described herein. The system 1200 can include at least one controller 1205, at least one pumps 410, at least one multiplexing switches 1215, and at least one electrodes 620. The controller can include at least one probe selector 1225, at least one connection establisher 1230, at least one measurement determiner 1235, at least one data manager 1240, at least one pump actuator 1245, and at least one sensor 1250. In some implementations, the at least one sensor 1250 can be external to and communicatively coupled to the controller 1205.

Each of the components (e.g., the probe selector 1225, the connection establisher 1230, the measurement determiner 1235, the data manager 1240, the pump actuator 1245, etc.) of the controller 1205 can be implemented using the hardware components or a combination of software with the hardware components of a computing system (e.g., computing system 1400, the controller 1205, any other computing system described herein, etc.) detailed herein in conjunction with FIG. 1400. Each of the components of the controller 1205 can perform the functionalities detailed herein.

The controller 1205 can include at least one processor and a memory, e.g., a processing circuit. The memory can store processor-executable instructions that, when executed by processor, cause the processor to perform one or more of the operations described herein.

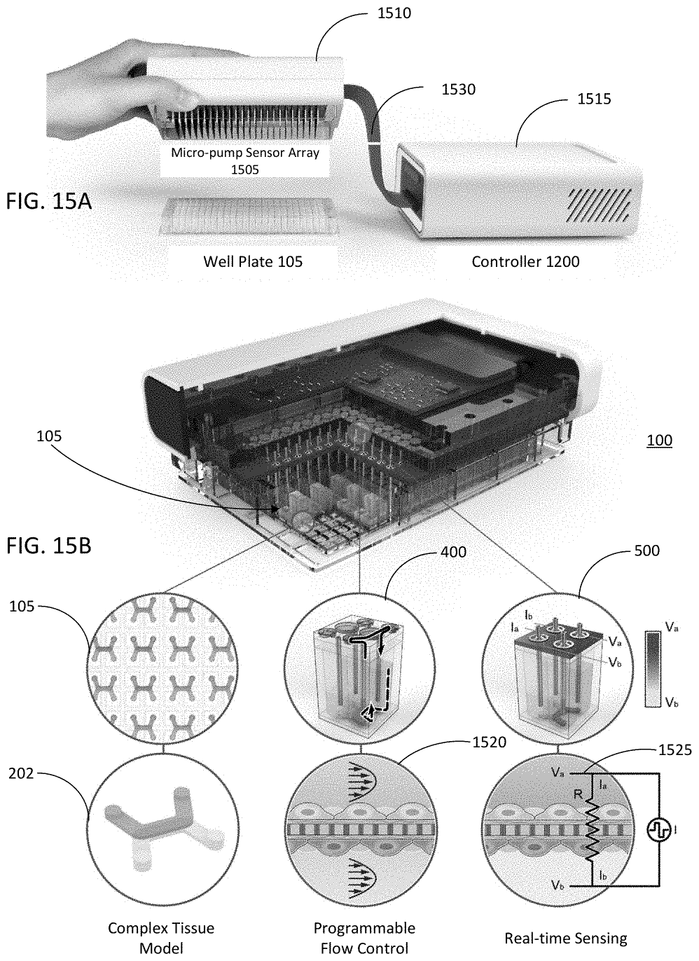

The processor may include a microprocessor, an application-specific integrated circuit (ASIC), a field-programmable gate array (FPGA), etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The controller 1205 can include one or more computing devices or servers that can perform various functions as described herein. The controller 1205 can include any or all of the components and perform any or all of the functions of the computer system 1400 described herein in conjunction with FIG. 14.

The sensor 1250 can be configured to provide a current source and sense voltage levels from at least two electrodes. The sensor 1250 can have one or more interfaces configured to couple to one or more electrodes, such as via a routing mechanism implemented by one or more multiplexing switches 1215. The sensor 1250 can be configured to provide a source of electric current via a current source electrode (e.g., which can be coupled to the sensor 1250 via routing techniques described herein, etc.). The current source electrode can provide a source of current from one electrode to another point in an electric circuit to which the sensor 1250 is connected. In some implementations, the sensor 1250 can create an electric current using at least two electrodes, where the path of the electric current flows from a first electrode to a second electrode, while passing through one or more fluids or cell cultures of a microfluidic device. The sensor 1250 can be configured to sense a voltage level, such as the voltage from at least one voltage sense electrode (e.g., which can be coupled to the sensor 1250 via routing techniques described herein, etc.) to another point in a circuit to which the sensor 1250 is electrically coupled. In some implementations, the sensor 1250 can measure a voltage level across two or more electrodes. For example, the two electrodes can be used to measure a voltage between two points that is created by the current source provided by the sensor 1250 (e.g., through a microfluidic device). Thus, the sensor 1250 can utilize the voltage signals and the current signals to determine a TEER measurement of the cells or fluids in a microfluidic device 202.

The multiplexing switches 1215 can be those that make up a part of the digital multiplex board 810. The multiplexing switches 1215 can be arranged such that each of the electrodes 620 (e.g., of the electrical routing board 700, etc.) is addressable by the controller 1205. Each of the multiplexing switches 1215 can be in communication with (e.g., receive digital signals from, etc.) the controller 1205 or the components thereof. For example, the multiplex switches 1215 can be arranged such that appropriate actuation of each switch (e.g., a digital selecting via addressing to turn the switch ON or OFF) can cause an electrically conductive path between the desired electrode 620 and the sensor 1250. Each of the multiplex switches 1215 can have at least two states: an ON state and an OFF state. In the ON state, the multiplex switch 1215 can conduct electricity (e.g., to create an electrical pathway). In the OFF state, the multiplex switch 1215 can be in a high-impedance state (e.g., does not create an electrical pathway). The multiplex switches 1215 can be connected to the controller 1205 and the electrodes 620 such that a proper combination of ON and an OFF state can cause a pathway to be created from each electrode 620 to the sensor 1250, which can be a part of or external to (e.g., attached via connector or other communications interface, etc.) the controller 1205 or the components thereof. In some implementations, the multiplex switches 1215 can be connected to the controller 1205 and the electrodes 620 such that a proper combination of ON and an OFF state can cause a pathway to be created from each group of four electrodes 620 (e.g., for a single TEER measurement of a microfluidic device 202, etc.) to the sensor 1250. The multiplex switches 1215 can change state in response to signals received from the controller 1205 or the components thereof.

The probe selector 1225 can receive a selection of a probe, for example via a user interface. The user interface can be via an external device having buttons and a screen, or can be provided on a personal computer in communication with the controller. The signals from the user interface can be communicated to the controller 1205, and can indicate a selection of a microfluidic device 202 from which to take a measurement. Because the controller 1205 can be coupled to many electrodes 620, which can be disposed within many different wells of a microfluidic device, the probe selector 1225 can receive a selection (e.g., coordinates of a desired microfluidic device 202 for measurement, etc.) of a microfluidic device and can determine which of the electrodes 620 correspond to (e.g., are disposed within the wells of, etc.) the selected microfluidic device 202 of the well plate 105. For example, a particular microfluidic device 202 may be associated with probes that are addressed using particular address values. The probe selector 1225 can maintain an association (e.g., in the memory of the controller 1205, etc.) between each microfluidic device 202 location and the address values of the probes that are disposed therein. Thus, the probe selector 1225 can utilize the selection of the microfluidic device 202 to retrieve the corresponding probe address values. The selection of the microfluidic device 202 can identify a well of a well plate 105, and vice versa. Thus, in some implementations, the probe selector 1225 can receive a selection of a well that indicates a selection of a microfluidic device 202, and probes that correspond thereto. The probe selector 1225 can thus utilize the identification of the well for measurement to retrieve corresponding probe address values, as above.

The connection establisher 1230 can utilize the probe address values to establish a connection between the probes of the selected microfluidic device 202 (e.g., or a well that corresponds thereto, etc.). To do so, the connection establisher 1230 can translate the probe address values into digital signals that the connection establisher can provide to one or more of the multiplex switches 1215, described herein above. For example, the address values of the individual probes can be provided by an address bus of the controller 1205 (e.g., and controlled at least in part by the connection establisher 1230, etc.) having a plurality of signals. Each of the plurality of signals can correspond to a respective one of multiplexing switches 1215. Thus, when the address values of the probes are provided on the address bus, the multiplexing switches 1215 are placed into a state that establishes a connection between the sensor 1250 and the electrodes 620. In implementations where the sensor 1250 is integrated with the controller 1205, the address values provided on the address bus cause the multiplexing switches 1215 to be placed into a state that establishes a connection between the sensor 1250 and the controller 1205 (e.g., and the sensor 1250 thereof). Establishing a connection can include creating a pathway (e.g., routing) for electrical signals to travel to and from the electrodes 620. The electrical signals can include an electrical current from current source, or voltage levels across one or more electrodes 620. Thus, when a connection is established by the connection establisher 1230, the sensor 1250, or the controller 1205, can communicate with the selected microfluidic device 202 for analysis. Communication with an electrode 620 (e.g., or a probe including one or more electrodes, etc.) can include providing a source of electric current, providing a source of electric voltage, sensing a current, or sensing voltage.

The measurement determiner 1235 can determine a measurement using the connection(s) established to the one or more electrodes 620. After the electrodes 620 that correspond to the selected well or microfluidic device 202 have established a connection to the sensor 1250 or the controller 1205 can obtain a measurement, such as a TEER measurement, using the electrodes. In some implementations, the sensor 1250 can perform some or all of the functionality of the measurement determiner 1235. To obtain a TEER measurement, the measurement determiner can select one or more of the selected electrodes 620 (e.g., the electrodes 620 that have an established connection with the controller 1205 or the sensor 1250, etc.) and route an electric current through the one or more probes. The probes selected as current source probes for the selected well or microfluidic device 202 can be selected such that a path taken by electricity will pass through the cell cultures of the microfluidic device 202, thereby creating a difference in voltage potential across the overlapping region 320 of the microfluidic device 202. Thus, the measurement determiner 1235 can route an electric current through the overlapping region 320 of the selected microfluidic device 202, and any cells or fluids contained therein.

The measurement determiner 1235 can utilize one or more other probes to measure the difference in voltage potential across the overlapping region 320 induced at least in part by the current source routed through the selected electrodes 620. To measure the voltage drop across the overlapping region 320, the measurement determiner 1235 can utilize the one or more probes that are disposed within the selected microfluidic device 202 that are not utilized as current source probes as voltage sense probes. The voltage sense probes can measure the voltage of a point in the microfluidic device with respect to a different point that is part of an electrical circuit to which the measurement determiner is electrically coupled, such as a ground voltage. In some implementations, to measure the voltage from a point in the microfluidic device with respect to a different point in the microfluidic device, the measurement determiner 1235 can measure the voltage using at least two electrodes disposed within the selected microfluidic device 202. For example, the measurement determiner 1235 can measure the voltage potential between each probe.

Thus, the measurement determiner 1235 can utilize four probes to route an electric current across the cells in a microfluidic device and measure the voltage potential across the cells using the current. From these values and Ohms law (e.g., resistance equals voltage divided by current, etc.), the measurement determiner 1235 can determine the TEER of the cells in the selected microfluidic device 202. In some implementations, the sensor 1250, which can be external to but in communication with the controller 1205 or the multiplex switches 1215, can perform the same functionality to determine the TEER measurement of the cells. In such implementations, the sensor 1250 may provide various measurements (e.g., amount of current flowing through the cells, voltage measured across the cells, computed resistance or TEER values, etc.) to the measurement determiner 1235. The resistance or TEER value of the cells can be a parameter of the fluid sample contained by the microfluidic device 202. In some implementations, the sensor 1250 can record different information besides a TEER value using the selected electrodes 620, such as electrical impedance spectroscopy. Electrical impedance spectroscopy involves measuring the electrical resistance of the fluid sample (e.g., including the cells) as described above, but using alternating current at a range of frequencies, for example from 1 mHz to 10 MHz, or any range therein.

The data manager 1240 can store one or more measurements by the components of the system 1200 in a memory, such as an external memory or a memory that is internal to the device. For example, the data manager 1240 can access one or more data structures in a memory, and record or otherwise store the measurement values for the provided current, the measured voltage, the calculated or measured resistance, or any other values determined by the devices of system 1200. The data manager 1240 can store these values in association with an identifier of the selected well (or the selected microfluidic device 202) that was desired for measurement. Thus, the data manager 1240 can maintain one or more data records that include TEER information for each requested value. These TEER values can be retrieved, requested, or otherwise accessed by other external computing devices for further analysis. In implementations where electrical impedance spectroscopy is performed, the data manager 1240 can store the range of frequency values (e.g., and individual frequency values, etc.) in association with the calculated electrical resistance of the microfluidic device 202. In implementations where the sensor 1250 is external to the controller 1205, the sensor can be coupled to a different memory, and record the values as described herein in that memory in a similar process. The information about the measured microfluidic device 202 can be accessed by external computing devices for further analysis, for example data plotting, or assessing the barrier function of epithelial cells in the overlapping membrane of the microfluidic device 202.

In some implementations, prior to or during the measurement process, the pump actuator 1245 can actuate one or more of the pumps 410 that are attached to the pump sippers (which can be the electrodes 620) disposed within the microfluidic device 202 that is to be measured. The pump actuator 1245 can set each pump 410 to a control condition to prevent significant current leak through the conductive fluid in the pump 410, for example before a TEER measurement is taken. A current leak can be an undesirable path taken by current. If fluid is present in the pump, or other conductive material forms a different circuit that present an unfavorable path (e.g., a path for current that does not pass through a target area for measurements, or a path that is longer than desired and passes through undesired areas, etc.). In some implementations, if a current leak is detected (e.g., current draw on the electrodes exceeds a threshold, etc.), the pump actuator 1245 can set the pumps 410 of the microfluidic device 202 to a control condition. For example, such a pump setting could ensure that a pump 410 contains fluid but valves are closed to prevent current leak, or that a pump 410 is pumped to a dry condition to remove the conductive fluid, or a combination of these two settings. The pump actuator 1245 can be communicatively coupled to the pumps 410, and provide one or more signals to the pumps 410 to coordinate their operation with the other components of the controller 1205. Thus, the pump actuator 1245 can control the pumps and set them to various settings, and also control the levels of various fluids inside the microfluidic device 202 by causing the pumps to transport liquid through the wells of the well plate 105.

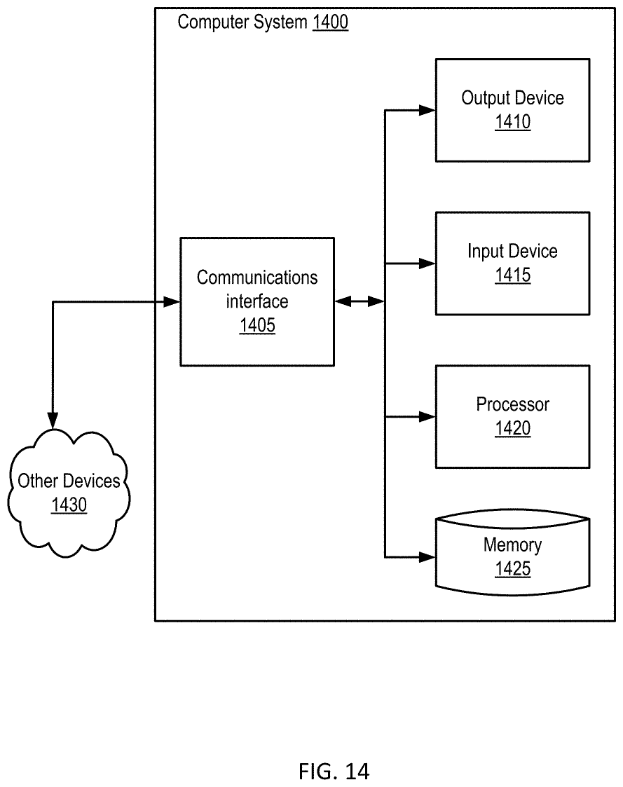

Referring now to FIG. 13, illustrated is an example method 1300 of establishing connections to probes and measuring properties of microfluidic devices. The method 1300 can be performed by any of the computing devices described herein, for example the controller 1205 described herein above in conjunction with FIG. 12, or the computing system 1400 described herein below in conjunction with FIG. 14. In brief overview of the method 1300, a controller device (e.g., the controller 1205, etc.) can receive an identification of a well of a well plate (e.g., the well plate 105, etc.) for measurement (STEP 1302), select a probe (e.g., one or more of the electrodes 620, etc.) that corresponds to the identification of the well for measurement (STEP 1304), establish a connection from a sensor (e.g., the sensor 1250, etc.) to a source electrode and a respective sense electrode of the probe (STEP 1306), determine a measurement of a fluid sample of a well using the sensor (STEP 1308), and store the measurement in a memory (STEP 1310).

In further detail of the method 1300, a controller device (e.g., the controller 1205, etc.) can receive an identification of a well of a well plate (e.g., the well plate 105, etc.) for measurement (STEP 1302). The controller device can receive a selection of a probe, for example via a user interface. The user interface can be via an external device having buttons and a screen, or can be provided on a personal computer in communication with the controller device. The signals from the user interface can be communicated to the controller device, and can indicate a selection of a microfluidic device of the well plate from which to take a measurement. Because the controller device can be coupled to many electrodes, which can be disposed within many different wells of various microfluidic devices in the well plate, the controller device can receive a selection (e.g., coordinates of a desired microfluidic device for measurement, etc.) of a microfluidic device and can determine which of the electrodes correspond to (e.g., are disposed within the wells of, etc.) the selected microfluidic device of the well plate.

The controller device can select a probe (e.g., one or more of the electrodes 620, etc.) that corresponds to the identification of the well for measurement (STEP 1304). The selected microfluidic device (or well) may be associated with probes (e.g., one or more electrodes, etc.) that are addressed using particular address values. The controller device can maintain an association (e.g., in the memory of the controller device, etc.) between each microfluidic device location and the address values of the probes that are disposed therein. Thus, the controller device can utilize the selection of the microfluidic device to retrieve the corresponding probe address values. The selection of the microfluidic device can identify a well of a well plate, and vice versa. Thus, in some implementations, the controller device can receive a selection of a well that indicates a selection of a microfluidic device, and probes that correspond thereto. The controller device can thus utilize the identification of the well for measurement to retrieve corresponding probe address values, as above.

The controller device can establish a connection from a sensor (e.g., the sensor 1250, etc.) to a source electrode and a respective sense electrode of the probe (STEP 1306). The controller device can utilize the probe address values to establish a connection between the probes of the selected microfluidic device (e.g., or a well that corresponds thereto, etc.). To do so, the controller device can translate the probe address values into digital signals that the controller device can provide to one or more of the multiplex switches (e.g., the multiplexing switches 1215, etc.) as described herein. For example, the address values of the individual probes can be provided by an address bus of the controller device (e.g., and controlled at least in part by the controller device, etc.) having a plurality of signals. Each of the plurality of signals can correspond to a respective one of multiplexing switches. Thus, when the address values of the probes are provided on the address bus, the multiplexing switches are placed into a state that establishes a connection between the sensor (e.g., the sensor 1250) and the electrodes. In implementations where the sensor is integrated with the controller device, the address values provided on the address bus cause the multiplexing switches to be placed into a state that establishes a connection between the sensor and the controller device (e.g., and the sensor thereof). Establishing a connection can include creating a pathway (e.g., routing) for electrical signals to travel to and from the electrodes. The electrical signals can include an electrical current from current source, or voltage levels across one or more electrodes. Thus, when a connection is established by the controller device, the sensor, or the controller device, can communicate with the selected microfluidic device for analysis. Communication with an electrode (e.g., or a probe including one or more electrodes, etc.) can include providing a source of electric current, providing a source of electric voltage, sensing a current, or sensing voltage.

The controller device can determine a measurement of a fluid sample of a well using the sensor (STEP 1308). After the electrodes that correspond to the selected well or microfluidic device have established a connection to the sensor or the controller can obtain a measurement, such as a TEER measurement, using the electrodes. In some implementations, the sensor can perform some or all of the functionality of the controller device. To obtain a TEER measurement, the measurement determiner can select one or more of the selected electrodes (e.g., the electrodes that have an established connection with the controller device or the sensor, etc.) and route an electric current through the one or more probes. The probes selected as current source probes for the selected well or microfluidic device can be selected such that a path taken by electricity will pass through the cell cultures of the microfluidic device, thereby creating a difference in voltage potential across the overlapping region (e.g., the overlapping region 320, etc.) of the microfluidic device. Thus, the controller device can route an electric current through the overlapping region of the selected microfluidic device, and any cells or fluids contained therein.

The controller device can utilize one or more other probes to measure the difference in voltage potential across the overlapping region induced at least in part by the current source routed through the selected electrodes. To measure the voltage drop across the overlapping region, the controller device can utilize the one or more probes that are disposed within the selected microfluidic device that are not utilized as current source probes as voltage sense probes. The voltage sense probes can measure the voltage of a point in the microfluidic device with respect to a different point that is part of an electrical circuit to which the measurement determiner is electrically coupled, such as a ground voltage. In some implementations, to measure the voltage from a point in the microfluidic device with respect to a different point in the microfluidic device, the controller device can measure the voltage using at least two electrodes disposed within the selected microfluidic device. For example, the controller device can measure the voltage potential between each probe.

Thus, the controller device can utilize four probes to route an electric current across the cells in a microfluidic device and measure the voltage potential across the cells using the current. From these values and Ohms law (e.g., resistance equals voltage divided by current, etc.), the controller device can determine the TEER of the cells in the selected microfluidic device. In some implementations, the sensor, which can be external to but in communication with the controller device or the multiplex switches, can perform the same functionality to determine the TEER measurement of the cells. In such implementations, the sensor may provide various measurements (e.g., amount of current flowing through the cells, voltage measured across the cells, computed resistance or TEER values, etc.) to the controller device. The resistance or TEER value of the cells can be a parameter of the fluid sample contained by the microfluidic device. In some implementations, the sensor can record different information besides a TEER value using the selected electrodes 620, such as electrical impedance spectroscopy. Electrical impedance spectroscopy involves measuring the electrical resistance of the fluid sample (e.g., including the cells) as described above, but using alternating current at a range of frequencies, for example from 1 mHz to 10 MHz, or any range therein.

The controller device can store the measurement in a memory (STEP 1310). The controller device can store one or more measurements in a memory, such as an external memory or a memory that is internal to the controller device. For example, the controller device can access one or more data structures in a memory, and record or otherwise store the measurement values for the provided current, the measured voltage, the calculated or measured resistance, or any other values determined by controller device or any devices in communication with the controller device (e.g., the sensor, etc.). The controller device can store one or more of these values in association with an identifier of the selected well (or the selected microfluidic device) that was desired for measurement. Thus, the controller device can maintain one or more data records that include TEER information for each requested value. These TEER values can be retrieved, requested, or otherwise accessed by other external computing devices for further analysis. In implementations where electrical impedance spectroscopy is performed, the controller device can store the range of frequency values (e.g., and individual frequency values, etc.) in association with the calculated electrical resistance of the microfluidic device. In implementations where the sensor is external to the controller device, the sensor can be coupled to a different memory, and record the values as described herein in that memory in a similar process. The information about the measured microfluidic device can be accessed by external computing devices for further analysis, for example data plotting, or assessing the barrier function of epithelial cells in the overlapping membrane of the microfluidic device.

FIG. 14 shows the general architecture of an illustrative computer system 1400 that may be employed to implement any of the computer systems discussed herein in accordance with some implementations. The computer system 1400 can be used to communicate and route signals between electrodes and a sensor to analyze the properties of fluids in the microfluidic device 202. The computer system 1400 of FIG. 14 comprises one or more processors 1420 communicatively coupled to memory 1425, one or more communications interfaces 1405, and one or more output devices 1410 (e.g., one or more display units) and one or more input devices 1415. The processors 1420 can be included in any of the computing device described herein.

In the computer system 1400 of FIG. 14, the memory 1425 may comprise any computer-readable storage media, and may store computer instructions such as processor-executable instructions for implementing the various functionalities described herein for respective systems, as well as any data relating thereto, generated thereby, or received via the communications interface(s) or input device(s) (if present). Referring again to the system 1400 of FIG. 14, the computer system 1400 can include the memory 1425 to store information any of the information, variables, vectors, data structures, or other computer-readable information described herein, among others. The processor(s) 1420 shown in FIG. 14 may be used to execute instructions stored in the memory 1425 and, in so doing, also may read from or write to the memory various information processed and or generated pursuant to execution of the instructions.

The processor 1420 of the computer system 1400 shown in FIG. 14 also may be communicatively coupled to or control the communications interface(s) 1405 to transmit or receive various information pursuant to execution of instructions. For example, the communications interface(s) 1405 may be coupled to a wired or wireless network, bus, or other communication means and may therefore allow the computer system 1400 to transmit information to or receive information from other devices 1430 (e.g., other computer systems, sensors, external storage devices, etc.). While not shown explicitly in the system of FIG. 14, one or more communications interfaces facilitate information flow between the components of the system 1400. In some implementations, the communications interface(s) may be configured (e.g., via various hardware components or software components) to provide a website as an access portal to at least some aspects of the computer system 1400. Examples of communications interfaces 1405 include user interfaces (e.g., web pages), through which the user can communicate with the computer system 1400.

The output devices 1410 of the computer system 1400 shown in FIG. 14 may be provided, for example, to allow various information to be viewed or otherwise perceived in connection with execution of the instructions. The input device(s) 1415 may be provided, for example, to allow a user to make manual adjustments, make selections, enter data, or interact in any of a variety of manners with the processor during execution of the instructions. Additional information relating to a general computer system architecture that may be employed for various systems discussed herein is provided further herein. While operations are depicted in the drawings in a particular order, such operations are not required to be performed in the particular order shown or in sequential order, and all illustrated operations are not required to be performed. Actions described herein can be performed in a different order.

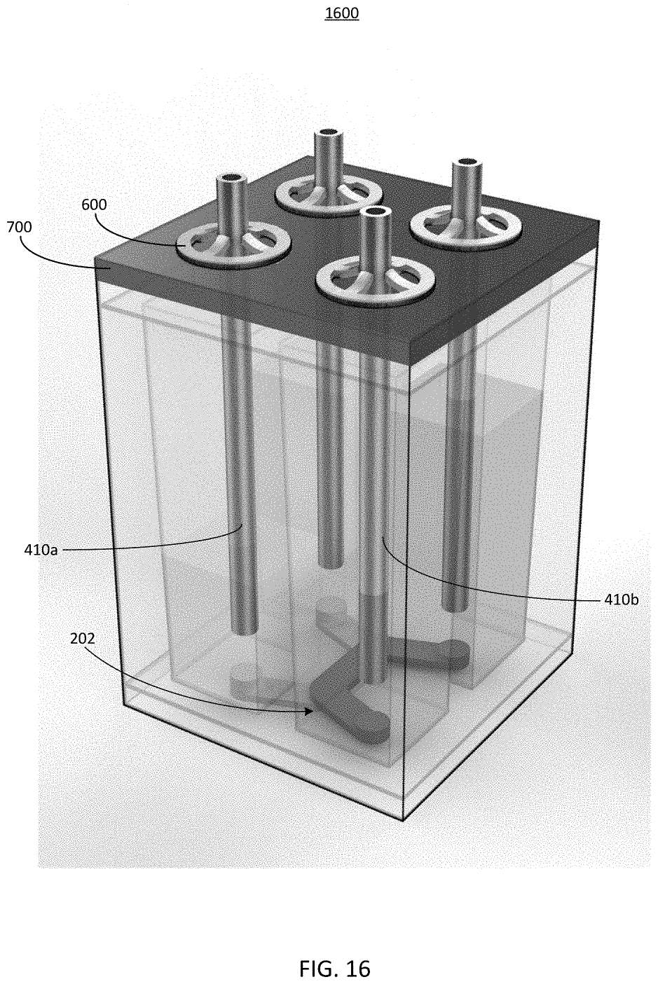

Referring now to FIG. 15A, depicted an example diagram of the various systems discuss herein, in accordance with one or more implementations. As depicted in FIG. 15A, in some implementations, the controller 1205 can be housed in a separate housing from the pump sippers 410 and the electrical routing board 700 as described herein in conjunction with FIGS. 4 and 7 respectively. The collective pumps, sippers, and electrical routing board can be implemented as a micro-pump sensor array (MPSA) 1505. As depicted in FIG. 15A and as described herein, the electrical routing board 700 can communicate with the controller 1205 via a high-density interconnect 1530, or other communications interface. In some implementations, the high density interconnect 1530 can be the same as or similar to the high-density interconnect 820 described herein above in conjunction with FIG. 8. The MPSA 1505 can have an insulated housing 1510, and the controller 1205 can have an insulated housing 1515, each of which can be similar to the housing 115 described herein above in conjunction with FIG. 1. Separating the controller 1205 from the MPSA 1505 can further isolate the controller 1205 from conductive fluids or other materials that may adversely affect the functionality of the controller 1205 of the components thereof as described herein.

Referring now to FIG. 15B, depicted is a sectional view of the system 100 described herein above as utilized for the various functionalities described herein. The system depicted in FIG. 15B can be the same as the system 100 described herein above in conjunction with FIG. 1, but including the depiction of the digital multiplexing board 810 and the components coupled thereto. FIG. 15B illustrates that the system 100 can implement teach of the functionalities described herein with respect to FIGS. 2, 4, and 5. In particular, the system 100 can be coupled with the well plate 105 to seed complex cell tissues in the microfluidic device 202. For example, the system 100 can utilize the pumps 110 to provide one or more fluid samples containing cells to the microfluidic devices 202 of the well plate 105, thereby precisely seeding cell cultures in the overlapping region 320 of the microfluidic device 202.

Further, the system 100 can implement the fluidic circuit 400 as described herein above in conjunction with FIG. 4. For example, the controller 1205 can actuate one or more of the pumps 410 using, for example, the pump actuator 1245. The fluidic circuit 400 can include microfluidic reservoirs 405 coupled with pumps 410. The microfluidic reservoirs can contain fluid samples that are introduced to the channels of the microfluidic device 202 by the pumps 410. The pumps 410 can have at least two ends, with one end coupled to at least one microfluidic reservoir and another end coupled to a portion of the microfluidic device 202, such as a port 304 or a port 314 described herein above in conjunction with FIG. 3. Thus, the pumps 410 can be coupled with a port of a well plate such as the well plate 105 shown in FIG. 1. For example, as depicted the pumps 410 may interface with a microfluidic device 202 similar to that shown in FIG. 2. In some implementations, each pump 410 may include a metal tube as depicted in FIG. 4, which can be referred to as a sipper.

The sipper of each pump can deliver or remove fluid from a respective well of the well plate 105. In some implementations, the pumps 410 can create a pressure in the well plate that causes one or more fluid samples to pass through at least one channel of the microfluidic device 202. The sipper of each pump 410 can deliver fluid from the reservoirs 405 to the well plate below. Further, the controller 1205 can utilize the pump actuator 1245 to implement programmable flow control inside the microfluidic device 202. For example, one or more of the pumps 410 can be actuated by the pump actuator 1245 to create a fluid pressure within the channels (e.g., the basal channel 302 or the apical channel 312, etc.) of the microfluidic device 202. The fluid pressure can be specified in one or more requests (e.g., from an external computer in communication with the controller 1205, etc.) that identify a well or a microfluidic device 202 that is coupled to or forms a part of the well plate 105. The request can specify a fluid pressure, one or more channels, and a flow direction to induce using the pumps 410 having sippers disposed within the microfluidic device 202 of the well plate 105. As described herein above, the pump actuator 1245 can provide one or more signals to the pumps 410 such that the requested fluid pressure or flow direction is induced within the desired channel of microfluidic device 202. In some implementations, a programmed fluid flow can be induced within more than one microfluidic device 202 of the well plate 105. In some implementations, a programmed fluidic flow can be induced within more than one channel of the specified microfluidic device 202. Thus, the pump actuator 1245 can create one or more programmed fluid flows 1520 within the microfluidic device, as described herein above.

As depicted in FIG. 15B, the system 100 can implement the example circuit 500 depicted in FIG. 5. For example, the circuit 500 can serve as a sensor (e.g., one or more probes gathering properties of a cells or fluid in a microfluidic device, etc.) for performing a measurement, such as a TEER measurement, on a microfluidic device 202. The circuit can comprise one or more electrodes inserted into the wells of the well plate 105 that are coupled to or form a part of the microfluidic device 202 under measurement (sometimes referred to as the target microfluidic device 202). In some implementations, the electrodes can be the sippers of the pumps 410 described herein above. To cause the electrodes to be part of the circuit 500, each of the electrodes can be routed (e.g., by the controller 1205 described herein below in conjunction with FIG. 12, etc.) using switching techniques to electrically coupled to a voltage sensor, a current sensor, a voltage source, or a current source, among others.

Generally, a TEER measurement can refer to an assay of the barrier function of the cultured cells within the microfluidic device 202 (e.g., the cells that are attached to and cultured on the overlapping region as described herein above, etc.). In this example, the TEER measurement can be achieved using a four point probe measurement which measures the electrical resistance of the tissue through providing two source electrodes and two sense electrodes. In the four point probe measurement, two of the electrodes can be current source electrodes, and two of the electrodes can be voltage sense electrodes. By changing the positioning (e.g., into which wells each electrode type is placed, etc.) of the current source electrodes and the voltage sense electrodes, different electrical paths are created through the microfluidic device 202. As shown in FIG. 15B, a respective electrode can be positioned to a respective well coupled to or forming a part of one of the four ports of the microfluidic device 202. In some implementations, the electrodes can be submerged into the fluidically connected wells of the microfluidic device 202, which can contain an electrically conductive fluid such as the cell culture media or a buffered salt solution. The conductive fluid can aid in the creation of a path for current to flow through the cells, thus improving the accuracy of the TEER measurements. Thus, the circuit 1525 can be created to determine the resistance R of the cells in the microfluidic device 202, using the features described herein above.

FIG. 16 illustrates a perspective view of a fluidic circuit 1600 that can be used in one or more of the systems described herein, in conjunction with one or more connectors for pump sippers, in accordance with an illustrative embodiment. The fluidic circuit 1600 can be an alternate view of the fluidic circuit 400, wherein each of the pumps 410 (e.g., the sippers of those pumps 410 as depicted in FIG. 16) are each mechanically coupled to a respective connector 600, as described herein above in conjunction with FIGS. 6A and 6B. Further, the fluidic circuit 1600 can serve as the example circuit of FIG. 5, where each of the pumps 410 can serve as an electrode disposed within an opening of the microfluidic device 202 that is electrically coupled to a respective connector 600. As described herein above, signals can be routed via the electrical routing board 700 from a sensor 1250 to a respective connector by one or more of the components of the controller 1205.

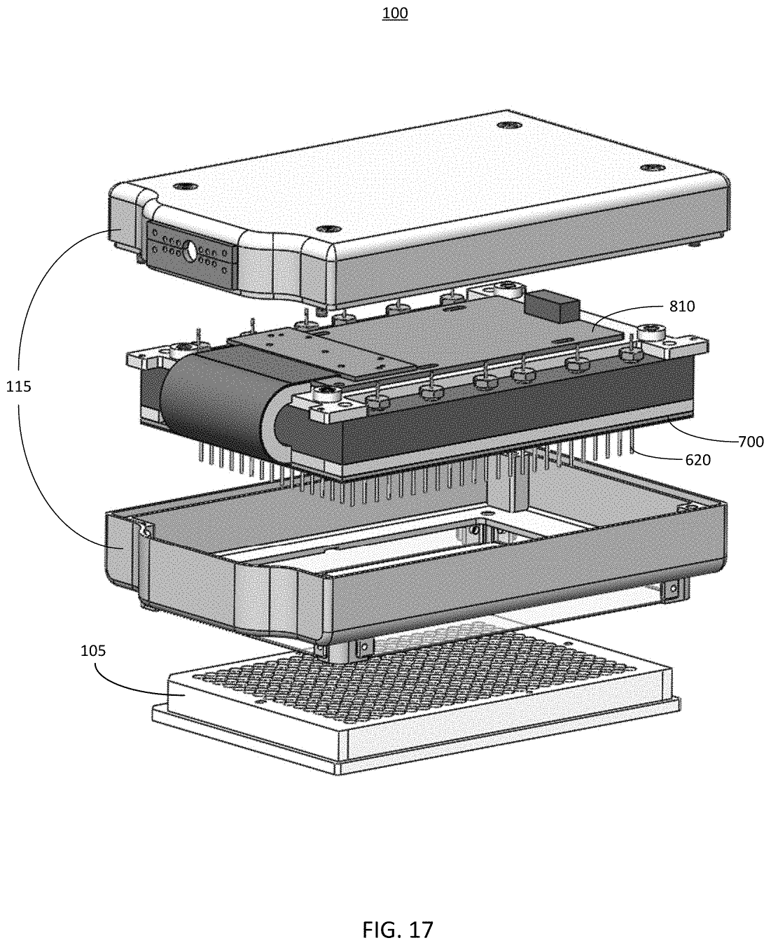

Referring now to FIG. 17, depicted is an expanded view of the system 100 including the features depicted in FIG. 9, in accordance with an illustrative embodiment. The housing 115 can be an enclosure that surrounds or partially surrounds other components of the system 1700 (e.g., which can be a. The system 100 can include a microfluidic well plate 105. The well plate 105 can include a plurality of wells, which may be interconnected by a network of channels within the well plate 105. The system 100 can also include a series of microfluidic pumps 110. Each pump 110 can be coupled with a respective port defined by the well plate 105. Thus, the pumps 110 can control the introduction of fluid samples into the wells of the well plate 105 via the ports with which the pumps 110 (not depicted) are coupled. Each of the pumps 110 can include a sipper 620 that can be disposed within a respective well of the well plate 105. Each of the pumps 110 can be electrically coupled to a controller, for example the controller 1205 described herein below in conjunction with FIG. 12. In some implementations, the controller 1205 can be disposed on the digital multiplexing board 810, or can be disposed on a different device (e.g., within the housing 1515 as depicted in FIG. 15, etc.) in communication with the digital multiplexing board 810. In such implementations, the housing 115 of the system 100 can thus be or be a part of the housing 1510 described herein above in conjunction with FIG.

15.

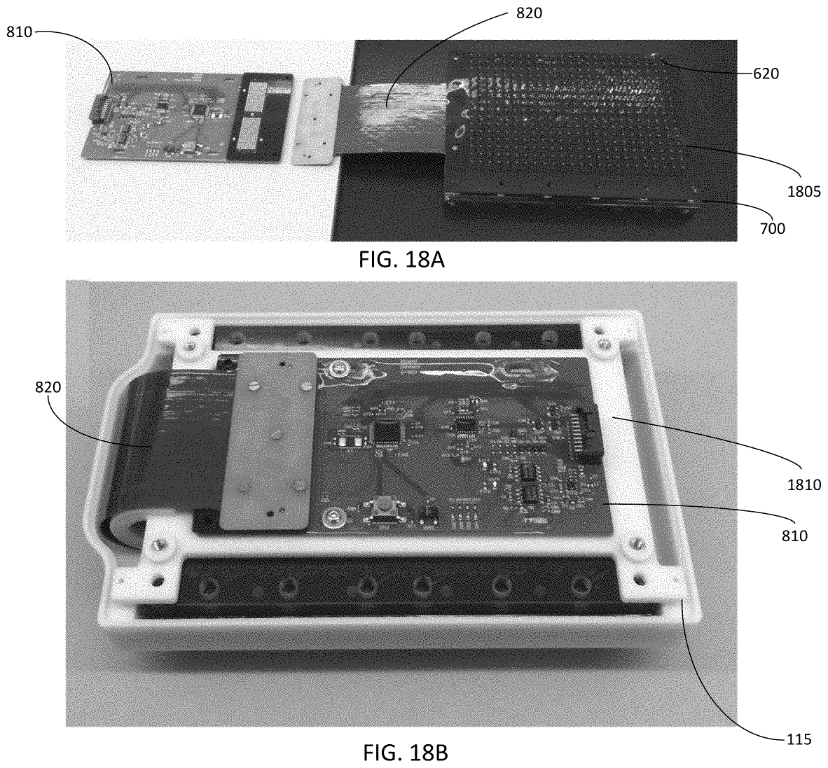

Referring now to FIGS. 18A and 18B, depicted are example implementations of portions of the systems described herein, in accordance with one or more implementations. For example, FIG. 18A depicts the digital multiplexing board 810, the high-density interconnect 820, one or more electrodes 620 (or pump sippers 620 as in some implementations, etc.) coupled to the electrical routing board 700. Although not pictured in FIG. 18A, it should be understood that each of sippers can be coupled to the electrical routing board 700 via one or more connectors 600 as described herein above. As depicted in FIG. 18A, the digital multiplexing board 810 can be separated from the electrical routing board 700, as the high-density interconnect can interface with the digital multiplexing board 810 via an interposer, as described herein above in conjunction with FIG. 8. Thus, the digital multiplexing board, or the electrical routing board 700, can be part of a modular design, and thus easily replaced if needed. Each of the electrodes 620 can be inserted into the wells of a well plate, such as the well plate 105. The electrical routing board can be electrically isolated from the portion of the electrodes 620 that are inserted into the wells of the well plate by the insulating layer 1805. The insulating layer 1805 can comprise any time of electrical insulator, such as a rubber compound, a plastic polymer, or other type of electrical insulator. In some implementations, the insulating layer 1805 can be made of a material that can keep any fluids provided by the pump sippers 620 from contacting the electrical routing board 700.

FIG. 18 depicts the digital multiplexing board 810 coupled to the electrical routing board 700 via the high-density interconnect 820. To further isolate the digital multiplexing board 810 from the electrical routing board 700 and from the well plate in which the pump sippers 620 are to be disposed, an additional separation layer 1810 can separate the digital multiplexing board from the other components of the system. The insulating layer 1810 can comprise any time of electrical insulator, such as a rubber compound, a plastic polymer, or other type of electrical insulator. In some implementations, the insulating layer 1810 can be coupled to or form a part of the housing 115. In some implementations, the insulating layer 1810 can be made of a material that can keep any fluids provided by the pump sippers 620 from contacting the electrical routing board 700.

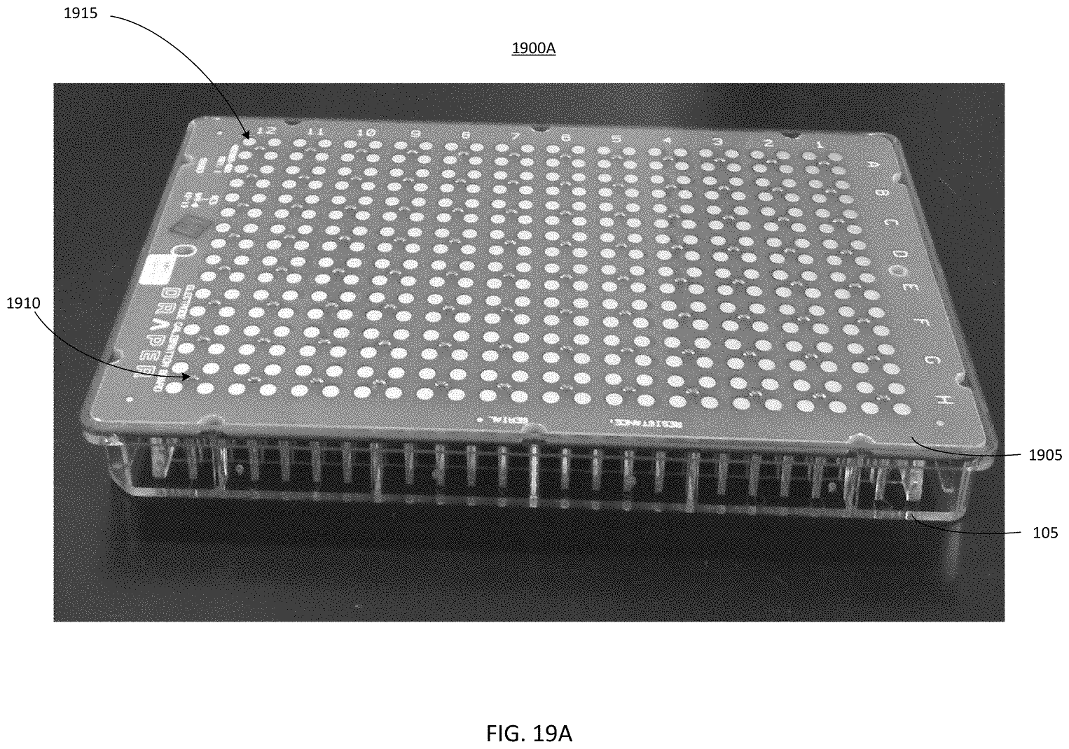

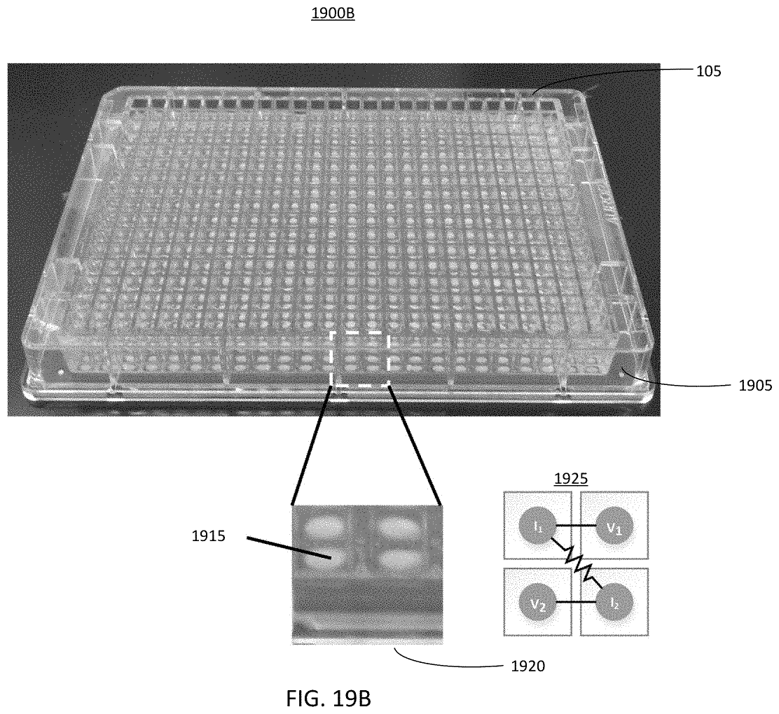

Referring now to FIG. 19A depicts a bottom view 1900A of an example implementation of a well plate 105 coupled with a calibration board 1905 for sensor 1250 calibration, in accordance with one or more implementations. To calibrate a sensor 1250, such as a sensor 1250 that can determine a TEER measurement, a known resistor value can be used. Because the wells of a well plate 105 that contain fluid samples may vary in resistance, a calibration board 1905 can be used to emulate a well plate configuration, but with known resistance values. Instead of having a microfluidic device 202 coupled to or forming a part of the bottom of each group of four wells, the well plate can instead have a calibration board 1905 that forms the bottom of the well plate 105. The calibration board 1905 can have a respective electrical pad 1915 that is exposed within the bottom of each well of the well plate 105. A known resistor 1910 (e.g., an accurate resistor having a known value, etc.) can be disposed in the middle of each group of four conductive pads 1915, each of which correspond to a respective well of the well plate 105. Each node of the known resistor 1910 can be electrically coupled to a respective two of the group of four conductive pads 1915, for example via one or more traces on the calibration board that being at a node of a resistor and terminate at an electric pad 1915. Thus, the known resistor can emulate the overlapping region 320 of a microfluidic device 202, but with a known resistance. Using the known resistance, the sensor 1250 can be calibrated using one or more sensor calibration techniques.

FIG. 19B depicts a top view 1900B of an example implementation of a well plate 105 coupled with a calibration board 1905 for sensor calibration, in accordance with one or more implementations. As described above, the calibration device 1920 (e.g., each group of four conductive pads 1915 and a known resistor used to emulate a microfluidic device 202, etc.) can be connected to the wells of a multi-well plate 105 in a 4-point probe configuration. As shown in FIG. 19B, each calibration device 1920 can correspond to a group of four wells in the well plate 105. The known resistor can be assembled on the opposing side of the wells adhered to a printed circuit board. Each of the conductive pads 1915 facing into the well plate can be electrically coupled to a respective pad 1915 on the opposite side of the calibration board 1905, for example via one or more traces or vias. The wells of the well plate 105 can be filled (e.g., using one or more pumps 110 controlled by the pump actuator 1945 of the controller 1205, etc.) with an ionic solution such as a salt buffer. The electrodes 620 that are communicatively coupled with one or more sensors 1250, as described herein above, can be inserted into the wells to complete the measurement circuit. Thus, the calibration board 1905 coupled to the well plate 105 can allow for the calibration of the sensor 1250 with a known resistor values, in a way that emulates the microfluidic devices 202 as described herein. To calibrate the sensor 1250, the sensor can utilize two electrodes 620 as current sources and two electrodes 620 as voltage sense probes, and thus create the circuit 1925 depicted in FIG. 19B. The readings of the sensor 1250 can be adjusted, either automatically by the controller 1205 or manually by a user, to match the expected known value of the known resistor 1910 using an offset value. In some implementations, the controller 1205 can store the offset value in one or more data structures in the memory of the controller 1205.

The separation of various system components does not require separation in all implementations, and the described program components can be included in a single hardware or software product.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the terms “about” and “substantially” will be understood by persons of ordinary skill in the art and will vary to some extent depending upon the context in which they are used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation or embodiment, and references to “an implementation,” “some implementations,” “one implementation,” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description, or any claim are followed by reference signs, the reference signs have been included to increase the intelligibility of the drawings, detailed description, and claims. Accordingly, neither the reference signs nor their absence has any limiting effect on the scope of any claim elements.

The devices, systems, and methods described herein may be embodied in other specific forms without departing from the characteristics thereof. The foregoing implementations are illustrative rather than limiting of the described devices, systems, and methods. Scope of the devices, systems, and methods described herein is thus indicated by the appended claims, rather than the foregoing description, and changes that come within the meaning and range of equivalency of the claims are embraced therein.

What is claimed is:

1. A system, comprising:
a well plate having one or more wells;
a plurality of probes having a source electrode and a respective sense electrode, the plurality of probes disposed within the one or more wells of the well plate; and
a controller device configured to:
receive an identification of a well of the well plate for a measurement;
select a probe of the plurality of probes that corresponds to the identification of the well for measurement;
establish a connection from a sensor to the source electrode and the respective sense electrode of the probe;
determine a measurement of a fluid sample in the well from the probe using the sensor; and
store the measurement in memory.

2. The system of claim 1, further comprising one or more pumps each disposed within a respective well of the one or more wells, wherein the one or more pumps are electrically coupled to and controlled by the controller device.

3. The system of claim 2, wherein the source electrode and the respective sense electrode are each coupled to a respective pump.

4. The system of claim 1, further comprising:
an airtight enclosure enclosing the controller device; and
a source of dry gas passing through the airtight enclosure to remove moisture from the controller device.

5. The system of claim 1, wherein the one or more wells of the well plate comprise up to 96 wells arranged in a rectangular pattern.

6. The system of claim 3, wherein the controller device further comprises one or more connectors configured to couple the controller device to the one or more pumps through mechanical force.

7. The system of claim 6, wherein the one or more connectors comprise at least one of a spring device, a conductive flexible material, or a deformable material that is pushed into place against the one or more pumps.

8. The system of claim 1, wherein the source electrode and the sense electrode of each of the one or more probes comprises at least one of silver, silver chloride, platinum, stainless steel, a polyimide polymer, or a polyether imide polymer.

9. The system of claim 1, further comprising:
a printed circuit board in electrical communication with the controller device and the one or more probes, comprising one or more multiplex switches; and
wherein the controller device is further configured to establish the connection from a sensor to the source electrode and the respective sense electrode by addressing the selected probe using the one or more multiplex switches.

10. The system of claim 6, wherein the one or more connectors each comprise:
an opening configured to receive a portion of a pump of the one or more pumps; and
a plurality of cantilevers arranged around the opening of the connector and configured to electrically couple the portion of the pump to the connector.

11. A method, comprising:
receiving, by a controller device in electrical communication with a plurality of probes, an identification of a well of a well plate for a measurement;
selecting, by the controller device, a probe of the plurality of probes that corresponds to the identification of the well for measurement;
establishing, by the controller device via one or more switches, a connection from a sensor to a source electrode and a respective sense electrode of the probe;
determining, by the controller device, a measurement of a fluid sample in the well using the sensor; and
storing, by the controller device, the measurement in a memory.

12. The method of claim 11, wherein selecting the probe of the plurality of probes that corresponds to the identification of the well for measurement further comprises selecting a second source electrode and a second respective sense electrode.

14. The method of claim 11, wherein establishing the connection from the sensor to the probe further comprises electrically coupling, by the controller device, the sensor to the source electrode and the respective sense electrode of the probe, such that the sensor can receive one or more signals from the probe.

15. The method of claim 11, wherein the sensor is integrated with the controller device, and determining the measurement of the fluid sample in the well further comprises:
providing, by the controller device using the source electrode of the probe, a current source from the probe of the plurality of probes that corresponds to the identification of the well to a second probe of the plurality of probes; and
sensing, by the controller device from the sense electrode of the probe, a voltage value between the sense electrode of the probe and the second probe of the plurality of probes.

16. The method of claim 15, wherein determining the measurement of the fluid sample comprises determining a resistance of a cell culture in the well using the voltage value.

17. A system, comprising:
a plurality of current source electrodes of a first respective plurality of pump sippers disposed within a first respective plurality of wells in a well plate;
a plurality of voltage sense electrodes of a second respective plurality of pump sippers disposed within a second respective plurality of wells, the second respective plurality of wells coupled to the first plurality of wells via a channel comprising a solution including cells; and
a controller device configured to:
route an electric current through the cells in the channel using a first current source electrode and a second current source electrode of the plurality of current source electrodes;
measure a voltage level across the cells caused in part by the electric current;
determine a parameter of the solution using the voltage level; and
actuate a pump of one of the first plurality of pump sippers or the second plurality of pump sippers.

18. The system of claim 17, wherein the controller device is configured to determine an impedance of the solution including the cells as the parameter.

19. The system of claim 17, wherein the controller device is configured to generate one or more signals to one or more switches to cause the electric current to flow through the first current source, the cells, and the second current source.

20. The system of claim 17, wherein the plurality of current source electrodes and the plurality of voltage sense electrodes comprise at least one of:
round wires;
flat wires;
conductive tubes; or
multi-lumen tubes.
